(19) United States
(12) Patent Application Publication
(10) Pub. No.: US 2014/0120063 A1
(43) Pub. Date: May 1, 2014

(54) NUTRITIONAL AND METABOLIC APPROACHES TO PREVENT EMERGENCE OF ENTERIC PATHOGENS

(71) Applicant: The Board of Trustees of the Leland Stanford Junior University, Palo Alto, CA (US)

(72) Inventor: Justin L. Sonnenburg, Redwood City, CA (US)

(21) Appl. No.: 14/046,817

(22) Filed: Oct. 4, 2013

Related U.S. Application Data
(60) Provisional application No. 61/711,167, filed on Oct. 8, 2012.
(60) Provisional application No. 61/783,693, filed on Mar. 14, 2013.

(51) Int. Cl. A61K 35/74; A61K 45/06

(52) U.S. Cl. CPC A61K 35/741; A61K 45/06
USPC 424/93.4

(57) ABSTRACT

Antibiotic-associated enteric pathogens are shown to increase in mucosal carbohydrate availability that occur upon disruption of the competitive ecosystem. Transient post-antibiotic increase in monosaccharides liberated by the resident microbiota from host mucus provides a window of opportunity for these pathogens to expand to densities sufficient to induce self-promoting host inflammation.

### FIELD OF THE INVENTION

The present invention relates generally to therapeutic formulations that alter nutrient availability to enteric pathogenic bacteria during and consequent to antibiotic administration.

### BACKGROUND OF THE INVENTION

The normal microbiota of humans is exceedingly complex, and varies by individual depending on genetics, age, sex, stress, nutrition and diet of the individual. It has been calculated that a human adult houses about 10¹² bacteria on the skin, 10¹⁰ in the mouth, and 10¹⁴ in the gastrointestinal tract. The latter number is far in excess of the number of eucaryotic cells in all the tissues and organs which comprise a human.

The microbiota of the gut perform many metabolic activities, and influence the physiology of the host. Bacteria make up the majority of the gut microbiota, although it includes anaerobic members of archaea and eukarya. The majority of these microbes are obligate anaerobes, and a small percentage facultative anaerobes. It is estimated that between 300 and 1000 different species live in the gut, however, it is known that a smaller number of species dominate. Most belong to either the Firmicutes or Bacteroidetes phyla. Common genera include: Bacteroides, Clostridium, Fusobacterium, Eubacterium, Ruminococcus, Peptococcus, Peptostreptococcus, Akkermansia, Faecalibacterium, Roseburia, Prevotella and Bifidobacterium. Species from the genus Bacteroides alone constitute about 30% of all bacteria in the gut, suggesting that this genus is especially important in the functioning of the host.

Some members of gut microbiota have enzymes that human cells lack for breaking down certain polysaccharides. Carbohydrates that humans cannot digest without bacterial help include certain starches, fiber, oligosaccharides and sugars that are not digested and absorbed in the upper portion of the GI tract, e.g. lactose in the case of lactose intolerance and sugar alcohols, mucus produced by the gut, and many types of complex dietary plant polysaccharides. Changing the numbers and species of gut microbiota can alter community function and interaction with the host.

Limited nutrients and high microbial densities within the gut are among the factors hypothesized to protect the host against invading microbes. Oral antibiotic use is one of the leading risk factors for disease associated with Salmonella spp. and Clostridium difficile, consistent with increased enteric vulnerability upon disruption of the resident microbiota. In addition, mouse models of S. typhimurium or C. difficile infection commonly require disruption of the intestinal microbiota with antibiotics to promote pathogen expansion within the lumen of the gut (i.e., emergence) and to initiate disease.

The mechanisms by which the microbiota prevents bacterial pathogen emergence and how microbiota disruption enables pathogens to circumvent these mechanisms are largely unknown. The present invention addresses this issue.

### SUMMARY OF THE INVENTION

The human intestine, colonized by a dense community of resident microbes, is a frequent target of bacterial pathogens. Undisturbed, this intestinal microbiota provides protection from bacterial infections. Conversely, disruption of the microbiota with oral antibiotics often precedes the emergence of several enteric bacterial pathogens. It is shown herein that antibiotic-associated enteric bacterial pathogens can employ a strategy of catabolizing microbiota-liberated mucosal carbohydrates during their emergence within the gut. Enteric pathogens of interest include Clostridium difficile, Salmonella typhimurium, Escherichia coli, etc.

The present invention provides methods and therapeutic formulations, in which an effective dose of an agent that limits altered nutrient availability in the gut following antibiotic-induced disruption of the normal microbiota is provided to an individual in need thereof. In providing such a nutrient limitation, post-antibiotic growth of enteric pathogens is reduced. In some embodiments, the altered nutrient is a mucosal sugar, e.g. a cleavage product of a mucosal sugar such as fucose, sialic acid, and the like. As used herein, an effective dose of an agent prevents undesirable pathogen growth following altered nutrient availability. Alternatively, an effective dose restores normal levels of nutrients concurrent with, or following antibiotic administration.

In some embodiments, an individual selected for treatment with the methods of the invention has a known infection with an enteric bacterial pathogen, including Clostridium difficile, Salmonella typhimurium, Escherichia coli, etc. In other embodiments, an individual selected for treatment with the methods of the invention has been treated with an antibiotic that disrupts normal gut microbiota; for example 1, 2, 3, 4 or more days following initiation of such antibiotic treatment. In other embodiments, an individual selected for treatment with the methods of the invention is commencing treatment with an antibiotic that disrupts normal gut microbiota; such an individual may be at risk of infection with an enteric bacterial pathogen, including Clostridium difficile, Salmonella typhimurium, Escherichia coli, etc.

In some embodiments nutrient availability is limited by inhibiting enzyme activity, e.g. inhibiting activity of an enzyme that increases nutrient availability following antibiotic administration, including oral antibiotic treatment. Antibiotics that disrupt normal gut microbiota may be broad spectrum antibiotics. The effect of the antibiotic on the normal gut microbiota may be tested by, for example, quantitation of microbes in fecal samples, where a disruptive antibiotic is one that, in the absence of the methods of the invention, results in an increase in enteric bacterial pathogens by at least 10-fold, at least 100-fold, at least 10³-fold or more, for example after about one to 3 days.

Specific examples of such enzymes include, without limitation, sialidase (neuraminidase); fucosidase; etc., as well as metabolic enzymes that produce metabolites which may be used as nutrients by other microbes, where the enzyme activity is present in the gut of the individual. Inhibitors include small molecules, nucleic acids, peptides and proteins, and the like. In some such embodiments the enzyme being inhibited is an enzyme expressed by gut microbiota, including without limitation species of Bacteroides, e.g. Bacteroides thetaiotamicron (Bt), Bacteroides fragilis, Bacteroides caccae, Bacteroides vulgatus, Bacteroides ovatus, Bacteroides stericoris, etc.

In some embodiments of the invention, a sialidase inhibitor is administered to an individual concurrent with, or following a course of antibiotics that disrupts normal gut microbiota. The sialidase inhibitor is administered in a dose and a formulation that is effective in inhibiting the activity of sialidase in the gut of the individual. For example, an inhibitor that has high inhibitory activity against sialidase enzymes present in the gut may be selected for use. Such an inhibitor may have low activity against viral sialidases. Alternatively, known sialidase inhibitors, e.g. oseltamivir, zanamivir, laninamivir, peramivir, etc. are administered in a dose and formulation appropriate for inhibition of sialidase in the gut. Formulations of interest include formulations in which the agent or a combination of agents is formulated in a pill, capsule, etc. that allows delivery of the active agent to the intestine.

A sialidase inhibitor may be selected or formulated for use in the methods of the invention, such that the inhibitor has substantially no bioavailability outside of the gut. Such inhibitors may have very low bioavailability outside of the gut when orally administered; may have very high first-pass metabolism so as to have very low systemic bioavailability; may be provided in a formulation that is released in the intestine and has a short half-life or low permeability so as to limit bioactivity to the gut; and the like.

In some embodiments, agents that limit nutrient availability in the gut following antibiotic-induced disruption of the normal microbiota are one or both of probiotics and prebiotics, which increase the non-pathogen utilization of the nutrient, i.e. compete with enteric pathogens for the nutrient. Probiotic agents may include non-pathogenic microorganisms that utilize the nutrient, e.g. sialic acid, fucose, acetic acid, succinate, etc. Prebiotic agents may comprise nutrients that enhance the growth of such organisms.

In other embodiments, agents that limit nutrient availability in the gut following antibiotic-induced disruption of the normal microbiota are inhibitors of the enteric-pathogen use of the nutrient, e.g. inhibitors of uptake and metabolism, enzymes that degrade the nutrient, etc., and include small molecules, nucleic acids, peptides and proteins, etc. Such inhibitors include, without limitation, sialic acid-use inhibitor, fucose-use inhibitor, enzymes that degrade free sialic acid, e.g., sialic acidlyase; and the like.

The invention provides compositions for therapeutic delivery of at least one agent that limits nutrient availability in the gut following antibiotic-induced disruption of the normal microbiota, including unit dose forms of the formulations suitable for administration to patients. Such formulations may include formulations in which the active agent is contained within a pill, capsule, etc, suitable for oral administration. In some embodiments the agent that limits nutrient availability is combined in a formulation with an antibiotic that disrupts normal microbiota.

The methods of the invention can be used for prophylactic as well as therapeutic purposes. As used herein, the term “treating” refers both to the prevention of infection and the treatment of an ongoing infection. In some embodiments the active agents of the invention are administered before increased growth of the enteric pathogens. In other embodiments, the active agents of the invention are administered after increased growth of the enteric pathogens. Evidence of pathogen growth and/or therapeutic effect may be monitored, for example, by quantitation of microorganisms present in a fecal sample, etc, as known in the art.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Bt facilitates S. typhimurium and C. difficile carbohydrate utilization during emergence. A) Schematic of mouse infection experiments. Germ-free, GF; B. thetaiotaomicron, Bt; S. typhimurium, St; C. difficile, Cd. B) S. typhimurium operons displaying significant differences in gene expression levels in vivo in the presence and absence of Bt, 5 days post-infection. Colors indicate the deviation of each gene's signal above (purple) and below (green) its mean expression value across all six in vivo samples and duplicate in vitro growths conducted in minimal medium. C) Induction of S. typhimurium nanE and fucI in cecal contents 1 day post-infection relative to growth in LB broth [n=9 and 4 for St and St+Bt, respectively]. D) Competitive index of wild-type St/St-ΔnanAΔfucI in Bt-monoassociated (St+Bt) and ex-germ-free (St) mice 1 day post-infection. Horizontal bars indicate the geometric means of CI values, and individual CI values are represented with dots [n=5/group]. E) Induction of C. difficile nan genes in cecal contents 3 days post-infection relative to growth in minimal medium containing 0.5% glucose [n=4/group]. F) C. difficile density in feces 1 day post-infection [n=4/group]. Error bars indicate SEM.

FIG. 2. Bt-liberated sialic acid promotes emergence of S. typhimurium and C. difficile. A) Levels of free sialic acid in cecal contents in GF and gnotobiotic mice monoassociated for 10 days [n=3, 3, 5, 5, respectively]. B) Fold change of expression of S. typhimurium nanE in cecal contents 1 day postinfection relative to growth in vitro [n=9, 4, 5, 5, respectively]. C) Induction of C. difficile nanE expression in cecal contents 3 days post-infection relative to growth in minimal medium containing 0.5% glucose [n=4/group]. D) C. difficile density in feces 1 day post-infection [n=5/group]. E) C. difficile density 1 day post-infection in feces of PBS or exogenous free sialic acid (SIA) treated mice. [n=4-5/group]. F) Induction of C. difficile nanE gene expression 1 day post-infection in feces of PBS or exogenous free sialic acid (SIA) treated mice relative to growth in minimal medium containing 0.5% glucose [n=5/group]. Error bars indicate SEM.

FIG. 3. S. typhimurium and C. difficile utilize mucin-derived monosaccharides resulting from antibiotic treatment of conventional mice. A) Levels of free sialic acid in cecal contents of conventional mice (CONV), antibiotic-treated mice 1 day (Ab D1) and 3 days (Ab D3) post-treatment [n=8, 9, 3, respectively]. B) Competitive index of wt S. typhimurium versus S. typhimurium mutants in cecal contents (St-ΔnanA) or feces (St-ΔnanAΔfucI) of antibiotic-treated conventional mice. Horizontal bars indicate the geometric means of CI values, and individual CI values are represented with dots [n=5 and 9, respectively]. C) Induction of C. difficile nanA and nanT expression in fecal samples 1 day post-infection of antibiotic-treated conventional mice relative to growth in minimal medium containing 0.5% glucose [n=4/group]. D) Density of wt C. difficile or a mutant deficient in sialic acid consumption (Cd-nanT) 3 days post-infection in feces of antibiotic-treated conventional mice. [n=10/group]. Error bars indicate SEM.

FIG. 4. Model of nutrient availability and pathogen expansion after antibiotic treatment. A) Before antibiotic treatment, resource partitioning is extremely efficient, and there are limited free monosaccharides in the lumen. A polysaccharide-degrading commensal species may not be able to use all monosaccharides it can liberate, but other commensals in the lumen consume remaining monosaccharides. B) After antibiotic treatment, nutrient networks are disrupted. Concentrations of some monosaccharides liberated before antibiotic treatment transiently increase due to a reduction in the organisms that are able to catabolize them. Additionally, some monosaccharides accumulate as a result of surviving polysaccharide-degrading anaerobes. C) Pathogens are able to exploit the free monosaccharide pool and expand in the intestinal lumen.

FIG. 5. COG classification of S. typhimurium genes with significantly altered expression in vivo. 42 of the 59 significantly altered genes could be assigned to COG categories. Functional COG categories with significant over-representation in the dataset of differentially expressed genes, compared to their representation in the S. typhimurium LT2 genome, were determined using a hypergeometric distribution; * denotes p<0.05, ** denotes p<0.01.

FIG. 6. S. typhimurium densities. Fecal densities of S. typhimurium were quantified from infected germ-free and Bt-colonized mice one day post-infection.

FIG. 7. Pathology induced by S. typhimurium and C. difficile during infection of gnotobiotic mice. Hematoxylin and eosin-stained sections of colon tissue from germ-free or Bt-associated gnotobiotic mice one day post-infection with either S. typhimurium (St) or C. difficile (Cd) showing that Bt does not significantly influence inflammation at this early stage of infection.

FIG. 8. Requirement for S. typhimurium nan and fuc operons for in vitro growth on sialic acid and fucose. A) S. typhimurium SL1344 encodes the genes necessary to catabolize sialic acid and fucose. The nan and fuc operons are pictured in their genomic context. B) Growth of wild-type S. typhimurium and mutant S. typhimurium strains in M9 minimal medium containing 0.5% glucose (M9+glucose), 0.5% sialic acid (M9+sialic acid), or 0.5% fucose (M9+fucose). OD600 measurements were taken every 30 minutes. C, D) ΔNanA E. coli (Keio strain JW3194) and wild-type and ΔNanA S. typhimurium SL1344 strains were grown aerobically in M9 minimal media containing 0.5% total carbohydrate. Cells were grown in (C) 0.5% glucose or 0.25% glucose and 0.25% sialic acid, and (D) 0.5% glycerol (gro) or 0.25% glycerol and 0.25% sialic acid. Optical densities were measured at indicated times.

FIG. 9. C. difficile nan operon expression in vitro. A) C. difficile encodes the genes necessary to catabolize sialic acid. The nan operon is pictured in its genomic context. B) Growth of C. difficile in minimal medium (MM) containing 0.5% glucose (MM+glucose) or 0.5% sialic acid (MM+sialic acid). OD600 measurements were taken every 30 minutes for 20 hours. C) Induction of C. difficile nan genes from growth in MM+sialic acid relative to MM+glucose at an OD600 of 0.5. tcdA and tcdB correspond to the Toxin A and Toxin B genes respectively, which are not differentially expressed between the two conditions. D) Growth of wild-type C. difficile (Cd) and nanT⁻ mutant C. difficile (Cd-nanT⁻) in minimal media (MM) containing 0.5% glucose, 0.5% sialic acid, or water. OD600 measurements were taken every 30 minutes.

FIG. 10. Density of Bt in monoassociated mice. Germ-free mice were monoassociated with either wild-type or Bt-ΔBT0455 strains of Bt. Fecal densities were evaluated at 6 and 9 days post-colonization.

FIG. 11. C. difficile density in feces. Fecal densities were quantified at (A) 1.5 days and (B) 2 days post-infection [n=4-5/group].

FIG. 12. C. difficile emergence with free sialic acid administration. C. difficile infected germ-free mice were administered phosphate buffered saline (PBS) or exogenous free sialic acid (SIA) as described in Methods. C. difficile density in feces 1 day post-inoculation is shown [n=4/group].

FIG. 13. Impact of antibiotic treatment on the intestinal microbiota. Conventional mice were gavaged with 20 mg streptomycin; bacterial densities and community composition were evaluated before (D0) and for specified time points post-treatment. A) Density of culturable bacteria using anaerobic and aerobic plating. B) Unweighted UniFrac-based principal coordinate analysis (PCoA) plot of fecal microbial communities determined by 16S rRNA enumeration as described in Methods. C) Weighted UniFrac-based principal coordinate analysis (PCoA) plot of fecal microbial communities determined by 16S rRNA enumeration as described in Methods. D) Taxa summary charts of 16S rRNA enumeration before (D0) and 1 (D1) or 5 (D5) days after antibiotic treatment.

### DEFINITIONS

Microbiota. As used herein, the term microbiota refers to the set of microorganisms present within or upon an individual, usually an individual mammal and more usually a human individual. Of particular interest is the microbiota of the gut. While the microbiota may include pathogenic species, in general the term references those commensal organisms found in the absence of disease. The gut microbiota of adult humans is primarily composed of obligate anaerobic bacteria.

In a healthy animal, while the internal tissues, e.g. brain, muscle, etc., are normally presumed to be free of microorganisms, the surface tissues, i.e., skin and mucous membranes, are constantly in contact with environmental organisms and become readily colonized by various microbial species. The mixture of organisms known or presumed to be found in humans at any anatomical site is referred to as the “indigenous microbiota”.

In humans, there are differences in the composition of the microbiota which are influenced by numerous factors including but not limited to age, diet, and the use of antibiotics. The microbiota of the large intestine (colon) is qualitatively similar to that found in feces. Populations of bacteria in the colon reach levels of 10¹¹/ml feces. The intestinal microbiota of humans is dominated by species found within two bacterial phyla: members of the Bacteroidetes and Firmicutes make up >90% of the bacterial population. Actinobacteria (e.g., members of the Bifidobacterium genus) and Proteobacteria among several other phyla are less prominently represented. Significant numbers of anaerobic methanogens (up to 10¹⁰/gm) may reside in the colon of humans. Common species of interest include prominent or less abundant members of this community, and may comprise, without limitation, Bacteroides thetaiotaomicron; Bacteroides caccae; Bacteroides fragilis; Bacteroides melaminogenicus; Bacteroides oralis; Bacteroides uniformis; Lactobacillus spp.; Clostridium perfringens; Clostridium septicum; Bifidobacterium bifidum; Enterococcus faecalis; Escherichia coli; Salmonella enteritidis; Klebsiella spp.; Enterobacter spp.; Proteus mirabilis; Pseudomonas aeruginosa; Peptostreptococcus spp.; Peptococcus spp., Faecalibacterium spp; Roseburia spp.; Ruminococcus spp.; Dorea spp.; Alistipes spp.; Akkermansia spp.; Prevotella spp. etc.

The composition of the microbiota of the gastrointestinal tract varies longitudinally along the tract (along the cephalocaudal axis) and transversely across the tract (with increasing distance from the mucosa). There is frequently a very close association between specific bacteria in the intestinal ecosystem and specific gut tissues or cells (evidence of tissue tropism and specific adherence). Gram-positive bacteria, such as the streptococci and lactobacilli, are thought to adhere to the gastrointestinal epithelium using polysaccharide capsules or cell wall teichoic acids to attach to specific receptors on the epithelial cells. Members of the segmented filamentous bacteria (SFBs) adhere to intestinal epithelium using a specialized structure on the cell surface known as a holdfast. Gram-negative bacteria such as the enterics may attach by means of specific fimbriae which bind to glycoproteins on the epithelial cell surface.

Prebiotic compounds. As used herein the term “prebiotic” refers to nutritional supplements that are not digested by the mammal that ingests them, but which are a substrate for the growth or activity of the microbiota, particularly the gut microbiota. Many prebiotics are carbohydrates, e.g. polysaccharides and oligosaccharides, but the definition does not exclude non-carbohydrates. The most prevalent forms of prebiotics are nutritionally classed as soluble fiber. Prebiotics may provide for changes in the composition and/or activity of the gastrointestinal microbiota. See Gibson and Roberfroid Dietary modulation of the human colonic microbiota: introducing the concept of prebiotics. J. Nutr. 1995 June; 125(6):1401-12, herein incorporated by reference.

As used herein, “probiotic” refers to microorganisms (e.g., bacteria, yeast, viruses and/or fungi) that form at least a part of the transient or endogenous microbiota and, thus, have a beneficial prophylactic and/or therapeutic effect on the host organism. Probiotics are generally known to be safe by those skilled in the art. The probiotic activity of a bacterial species may result from competitive inhibition of growth of pathogens due to superior colonization, parasitism of undesirable microorganisms, lactic acid production and/or other extracellular products having antimicrobial activity, or combinations thereof.

Sialidase inhibitor. A sialidase inhibitor, which may be a neuraminidase inhibitor, is an agent that inhibits the activity of a sialidase enzyme. In some embodiments the sialidase enzyme is one or a combination of enzymes that are active in the gut of an individual selected for treatment, particularly activity in the gut following antibiotic treatment that disrupts normal gut microflora. The sialidase may be host-derived, may be derived from gut commensals, e.g. Bacteroides species, or may be a combination thereof.

FDA approved neuraminidase inhibitors include oseltamivir (Tamiflu), zanamivir (Relenza; Glaxo Smith Kline, Research Triangle Park, N.C.), peramivir (BioCryst, Birmingham, Ala.), laninamivir; or the inhibitor may be a variant thereof. For example, the viral neuraminidase inhibitor, oseltamivir is an ethyl ester prodrug that can be purchased from Roche Laboratories (Nutley, N.J.). Amino acid sequences of FDA approved viral neuraminidase inhibitors can also be derivatized, for example, bearing modifications other than insertion, deletion, or substitution of amino acid residues, thus resulting in a variation of the original product.

Enzyme inhibitors. As discussed above, inhibitors of enzymes, e.g. sialidase inhibitors, fucosidase inhibitors, etc. find use in the methods of the invention. In addition to known small molecule inhibitors, inhibitors may include peptides, nucleic acids, novel small molecule inhibitors, and the like.

Inhibitors of interest include nucleic acids, e.g. which inhibit gene expression, including siRNA, interfering RNA or RNAi; dsRNA; RNA Polymerase III transcribed DNAs; ribozymes; and antisense nucleic acid, which can be RNA, DNA, or artificial nucleic acid. Also included are oligonucleotide sequences that include antisense oligonucleotides and ribozymes that function to bind to, degrade and/or inhibit the translation of an mRNA encoding a gut enzyme.

An aptamer can be nucleic acid ligand that, through its ability to adopt a specific three-dimensional conformation, binds to and has an antagonizing (i.e., inhibitory) effect on a target. The target of the present invention is a gut enzyme, e.g. sialidase, fucosidase, etc. Inhibition of the target by the aptamer can occur by binding of the target, by catalytically altering the target, by reacting with the target in a way which modifies/alters the target or the functional activity of the target, by covalently attaching to the target as in a suicide inhibitor, by facilitating the reaction between the target and another molecule. Aptamers can be comprised of multiple ribonucleotide units, deoxyribonucleotide units, or a mixture of both types of nucleotide residues. Aptamers can further comprise one or more modified bases, sugars or phosphate backbone units as described in further detail herein.

Aptamers nucleic acid sequences are readily made that bind to a wide variety of target molecules. The aptamer nucleic acid sequences of the invention can be comprised entirely of RNA or partially of RNA, or entirely or partially of DNA and/or other nucleotide analogs. Aptamers are developed to bind particular ligands by employing known in vivo or in vitro (more often, in vitro) selection techniques known as SELEX (Systematic Evolution of Ligands by Exponential Enrichment).

The term “antibiotic” as used herein includes all commonly used bacteristatic and bactericidal antibiotics, usually those administered orally. Antibiotics include aminoglycosides, such as amikacin, gentamicin, kanamycin, neomycin, streptomycin, and tobramycin; cephalosporins, such as cefamandole, cefazolin, cephalexin, cephaloglycin, cephaloridine, cephalothin, cephapirin, and cephradine; macrolides, such as erythromycin and troleandomycin; penicillins, such as penicillin G, amoxicillin, ampicillin, carbenicillin, cloxacillin, dicloxacillin, methicillin, nafcillin, oxacillin, phenethicillin, and ticarcillin; polypeptide antibiotics, such as bacitracin, colistimethate, colistin, polymyxin B; tetracyclines, such as chlortetracycline, demeclocycline, doxycycline, methacycline, minocycline, tetracycline, and oxytetracycline; and miscellaneous antibiotics such as chloramphenicol, clindamycin, cycloserine, lincomycin, rifampin, spectinomycin, vancomycin, and viomycin. Additional antibiotics' are described in “Remington's Pharmaceutical Sciences,” 16th Ed., (Mack Pub. Co., 1980), pp. 1121-1178.

Mucin glycans. Mucin glycans are typically built upon an N-acetylgalactosamine that is O-linked to serine and threonine residues of the mucin protein, and the most abundant are based on five different core structures. In both the intestinal mucins, repeated motifs containing galactose and N-acetylglucosamine are present and terminate with fucose and sialic acid residues.

The mucin glycans are a highly heterogenous mixture, comprising linear and branched oligosaccharides from about 2 to about 10 monosaccharide subunits in length, usually including galactose, N-acetylgalactosamine, fucose, glucose and N-acetylglucosamine, with heterogenous linkages within any one glycan, e.g. a mixture of α2-3, α2-6, α1-2, α1-3, α1-4, β1-3, β1-4, β1-6, etc.

Nutrients release from mucin glycans include fucose, sialic acid, N-acetylglucosamine, galactose, etc. Nutrients derived from mucin glycans also include short oligosaccharides of from about 2 to about 6 monosaccharide subunits in length, from about 4 to about 6 monosaccharide subunits in length, and the like.

### DETAILED DESCRIPTION OF THE PREFERRED EMBODIMENTS

Reference now will be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment.

Thus, it is intended that the present invention cover such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

The present invention provides methods and therapeutic formulations for limiting altered nutrient availability in the gut following antibiotic-induced disruption of the normal microbiota. In providing such a nutrient limitation, post-antibiotic growth of enteric pathogens is reduced. In some embodiments, the altered nutrient is a mucosal sugar, e.g. a cleavage product of a mucosal sugar such as fucose, sialic acid, and the like.

The agent that limits nutrient availability may be combined in a formulation with a second agent of the same or a different type, e.g. two or more sialidase inhibitors of differing specificity may be combined; a sialidase inhibitor may be combined with a fucosidase inhibitor; a sialidase inhibitor may be combined with one or both of a prebiotic and a probiotic; a sialidase inhibitor may be combined with sialic acid lyase; and the like.

The agent or combination of agents may also be combined with the antibiotic that disrupts normal gut microflora growth. During a course of antibiotic treatment, the agent that limits nutrient availability may be provided at the initiation of antibiotic treatment; shortly following antibiotic treatment, e.g. 1, 2, 3, 4, 5, 6, 7, or more days following treatment; or may be administered upon diagnosis of undesirable enteric pathogen growth.

The agents are incorporated into a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, gels, microspheres, etc. As such, administration can be achieved in various ways, usually by oral administration.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Agents, including small molecule inhibitors, aptamers, enzymes, and the like may be delivered to the gut lumen by genetically engineered microbes, which are administered orally or rectally. The engineered probiotic agent may be derived from a standard probiotic strain or from a known microbial resident strain.

Formulations are typically provided in a unit dosage form, where the term “unit dosage form,” refers to physically discrete units suitable as unitary dosages for human subjects, each unit containing a predetermined quantity of active agent in an amount calculated sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The term “unit dosage form,” refers to physically discrete units suitable as unitary dosages for human subjects, each unit containing a predetermined quantity of agent in an amount calculated sufficient to produce the desired effect in association with a pharmaceutically acceptable diluent, carrier or vehicle. The specifications for the unit dosage forms of the present invention depend on the particular combination employed and the effect to be achieved, and the pharmacodynamics associated with the host.

In one aspect, the agents are formulated into pharmaceutical compositions by combination with appropriate, pharmaceutically acceptable carriers or diluents, and are formulated into preparations in solid, semi-solid, liquid, suspension, emulsion, or gaseous forms, such as tablets, capsules, powders, granules, ointments, solutions, suspensions, emulsions, suppositories, injections, inhalants, gels, microspheres, and aerosols. As such, administration of the active agent and/or other compounds can be achieved in various ways, usually by oral administration. In pharmaceutical dosage forms, the active agents may be administered in the form of their pharmaceutically acceptable salts, or they may also be used alone or in appropriate association, as well as in combination with other pharmaceutically active compounds. The following methods and excipients are exemplary and are not to be construed as limiting the invention.

Pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Exemplary organic bases are isopropylamine, diethylamine, ethanolamine, trimethylamine, dicyclohexylamine, choline and caffeine.

Although not required, oral formulations may optionally comprise enteric coatings, so that the active agent is delivered to the intestinal tract. A number of methods are available in the art for the efficient delivery of enterically coated proteins into the small intestinal lumen. Most methods rely upon release as a result of the sudden rise of pH when food is released from the stomach into the duodenum, or upon the action of pancreatic proteases that are secreted into the duodenum when food enters the small intestine. Exemplary films are cellulose acetate phthalate, polyvinyl acetate phthalate, hydroxypropyl methylcellulose phthalate and hydroxypropyl methylcellulose acetate succinate, methacrylate copolymers, and cellulose acetate phthalate. Other enteric formulations comprise engineered polymer microspheres made of biologically erodable polymers, which display strong adhesive interactions with gastrointestinal mucus and cellular linings and can traverse both the mucosal absorptive epithelium and the follicle-associated epithelium covering the lymphoid tissue of Peyer's patches. The polymers maintain contact with intestinal epithelium for extended periods of time and actually penetrate it, through and between cells. See, for example, Mathiowitz et al. (1997) Nature 386 (6623): 410-414. Drug delivery systems can also utilize a core of superporous hydrogels (SPH) and SPH composite (SPHC), as described by Dorkoosh et al. (2001) J Control Release 71(3):307-18.

Various methods for administration may be employed, preferably using oral administration. The dosage of the therapeutic formulation can vary widely, depending upon the nature of the disease, the frequency of administration, the manner of administration, the clearance of the agent from the host, and the like.

### Compound Screening

Screening techniques, for example as known in the art, may be used to identify agents that selectively or specifically inhibit enzymes present in the gut that, upon antibiotic treatment, result in increased nutrients that increase growth of enteric pathogens. Any screening technique known in the art can be used to screen for agonist or antagonist molecules (such as sialidase inhibitors) directed at a target of interest (e.g. a sialidase, fucosidase, etc.). The present invention encompasses screens for small molecule ligands or ligand analogs and mimics, as well as screens for natural ligands that bind to and antagonize target enzymes.

Test compounds may be screened from large libraries of synthetic or natural compounds. Numerous means are currently used for random and directed synthesis of saccharide, peptide, and nucleic acid based compounds. Synthetic compound libraries are commercially available or via collaborators. A rare chemical library is available from Aldrich (Milwaukee, Wis.). Alternatively, libraries of natural compounds in the form of bacterial, fungal, plant and animal extracts are available, or are readily producible. Additionally, natural and synthetically produced libraries and compounds are readily modified through conventional chemical, physical, and biochemical means.

Methods for preparing libraries of molecules are well known in the art and many libraries are commercially available. Libraries of interest in the invention include peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties, which are less subject to enzymatic degradation compared to their naturally-occurring counterparts. Libraries are also meant to include for example but are not limited to peptide-on-plasmid libraries, polysome libraries, aptamer libraries, synthetic peptide libraries, synthetic small molecule libraries and chemical libraries. The libraries can also comprise cyclic carbon or heterocyclic structure and/or aromatic or polyaromatic structures substituted with one or more of the above-identified functional groups.

Small molecule combinatorial libraries can also be generated. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A compound array can be a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address.

Methods for preparing or identifying peptides that bind to a particular target are known in the art. Molecular imprinting, for instance, can be used for the de novo construction of macromolecular structures such as peptides that bind to a particular molecule. One method for preparing inhibitors involves the steps of: (i) polymerization of functional monomers around a known substrate that exhibits a desired activity; (ii) removal of the template molecule; and then (iii) polymerization of a second class of monomers in, the void left by the template, to provide a new molecule which exhibits one or more desired properties which are similar to that of the template. In addition to preparing peptides in this manner other binding molecules such as polysaccharides, nucleosides, drugs, nucleoproteins, lipoproteins, carbohydrates, glycoproteins, steroids, lipids, and other biologically active materials can also be prepared. Other methods for designing such molecules include for example drug design based on structure activity relationships, which require the synthesis and evaluation of a number of compounds and molecular modeling.

A sialidase inhibitor according to the method of the invention modulates the activity of a gut sialidase by reducing the activity of the sialidase in the gut.

Demonstration of in vivo efficacy of the small molecule inhibitor may be assessed in a mouse model as described in the Examples, and may be accompanied by characterization of host responses to the change in microbiota composition. Additional tests may be dictated by the existing data regarding host health and disease status related to the bacterial species being studied. In diseases where therapeutic potential is supported, the inhibitor that allowed a basic question to be addressed in an animal model will serve as a candidate for drug development.

The following examples describe exemplary embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples all percentages are given on a weight basis unless otherwise indicated.

These and other modifications and variations to the present invention may be practiced by those of ordinary skill in the art, without departing from the spirit and scope of the present invention, which is more particularly set forth in the appended claims. In addition, it should be understood that aspects of the various embodiments might be interchanged both in whole and in part. Furthermore, those of ordinary skill in the art will appreciate that the foregoing description is by way of example only, and is not intended to limit the invention so further described in such appended claims. Therefore, the spirit and scope of the appended claims should not be limited to the description of the preferred versions contained therein.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

### Example 1

The human intestine, colonized by a dense community of resident microbes, is a frequent target of bacterial pathogens. Undisturbed, this intestinal microbiota provides protection from bacterial infections. Conversely, disruption of the microbiota with oral antibiotics often precedes the emergence of several enteric pathogens (Doorduyn, Y., et al. Epidemiology and infection 134, 617-626 (2006)) (Pavia, A. T. et al. The Journal of infectious diseases 161, 255-260 (1990)) (Pepin, J. et al. Clin Infect Dis 41, 1254-1260 (2005)) (Kelly, C. P., et al. N Engl J Med 330, 257-262 (1994)). How pathogens capitalize upon the failure of microbiota-afforded protection is largely unknown. Here we show that two antibiotic-associated pathogens, Salmonella typhimurium and Clostridium difficile, employ a common strategy of catabolizing microbiota-liberated mucosal carbohydrates during their emergence within the gut. S. typhimurium accesses fucose and sialic acid within the lumen of the gut in a microbiota-dependent manner, and genetic ablation of the respective catabolic pathways reduces its competitiveness in vivo. Similarly, C. difficile emergence is aided by microbiota-induced elevation of sialic acid levels in vivo. Colonization of gnotobiotic mice with a sialidase-deficient mutant of the model gut symbiont Bacteroides thetaiotaomicron (Bt) reduces free sialic acid levels resulting in a downregulation of C. difficile's sialic acid catabolic pathway and impaired emergence. These effects are reversed by exogenous dietary administration of free sialic acid. Furthermore, antibiotic treatment of conventional mice induces a spike in free sialic acid and mutants of both Salmonella and C. difficile that are unable to catabolize sialic acid exhibit impaired emergence. These data show that antibiotic-induced disruption of the resident microbiota and subsequent alteration in mucosal carbohydrate availability are exploited by these two distantly related enteric pathogens in a similar manner. This insight suggests new possibilities for therapeutic approaches for preventing diseases caused by antibiotic-associated pathogens.

The intestinal microbiota is composed of trillions of microbial cells that together form a complex, dynamic, and highly competitive ecosystem. Limited nutrients and high microbial densities likely play a key role in protecting the host against invading microbes (Stecher, B. et al. PLoS pathogens 6, e1000711 (2010)). Carbohydrates derived from diet or host play a well-established role in sustaining the resident members of the microbiota (Chang, D. E. et al. PNAS 101, 7427-7432 (2004)) (Sonnenburg, J. L. et al. Science 307, 1955-1959 (2005)) (Martens, E. C., et al. Cell host & microbe 4, 447-457 (2008)), and more recently have been shown to play important roles in gut microbiota-pathogen dynamics (Fabich, A. J. et al. Infection and immunity 76, 1143-1152 (2008)) (Kamada, N. et al. Science 336, 1325-1329 (2012)) (Pacheco, A. R. et al. Nature 492, 113-117 (2012)) (Maltby, R., et al. PLoS ONE 8, e53957 (2013)). Oral antibiotic use is one of the leading risk factors for disease associated with Salmonella spp. and Clostridium difficile, consistent with increased enteric vulnerability upon disruption of the resident microbiota. In addition, mouse models of S. typhimurium or C. difficile infection commonly require disruption of the intestinal microbiota with antibiotics to promote pathogen expansion within the lumen of the gut (i.e., emergence) and to initiate disease (Hapfelmeier, S., et al. Trends in microbiology 13, 497-503 (2005)) (Lawley, T. D. et al. Infection and immunity 76, 403-416 (2008)) (Lawley, T. D. et al. Infection and immunity 77, 3661-3669 (2009)) (Chen, X. et al. Gastroenterology 135, 1984-1992 (2008)). Deciphering the numerous mechanisms by which the microbiota prevents bacterial pathogen emergence and how microbiota disruption enables pathogens to circumvent these mechanisms remains an important task.

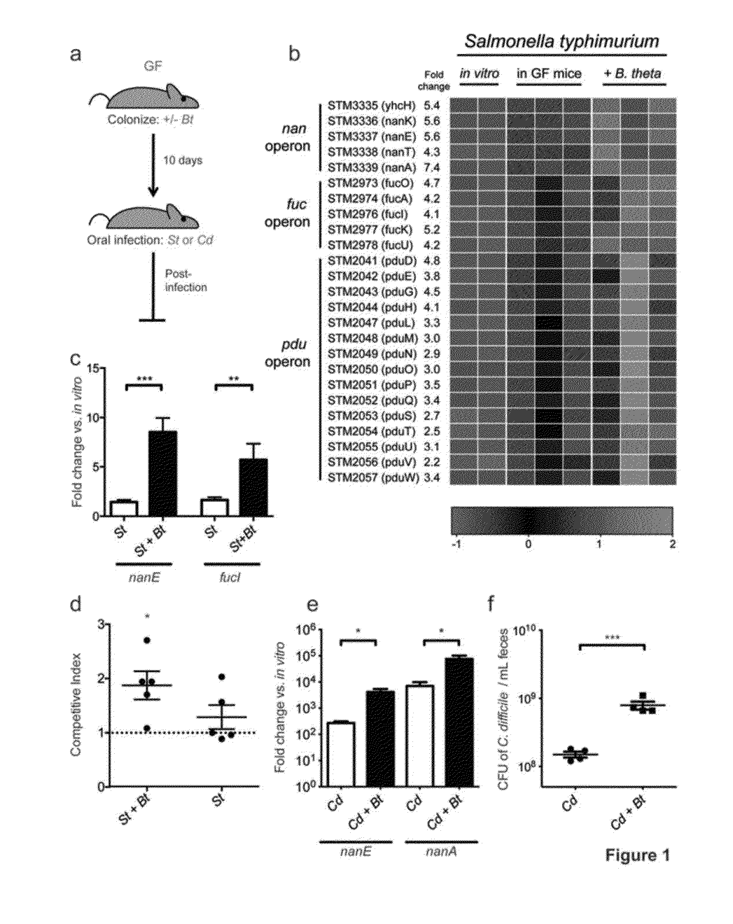

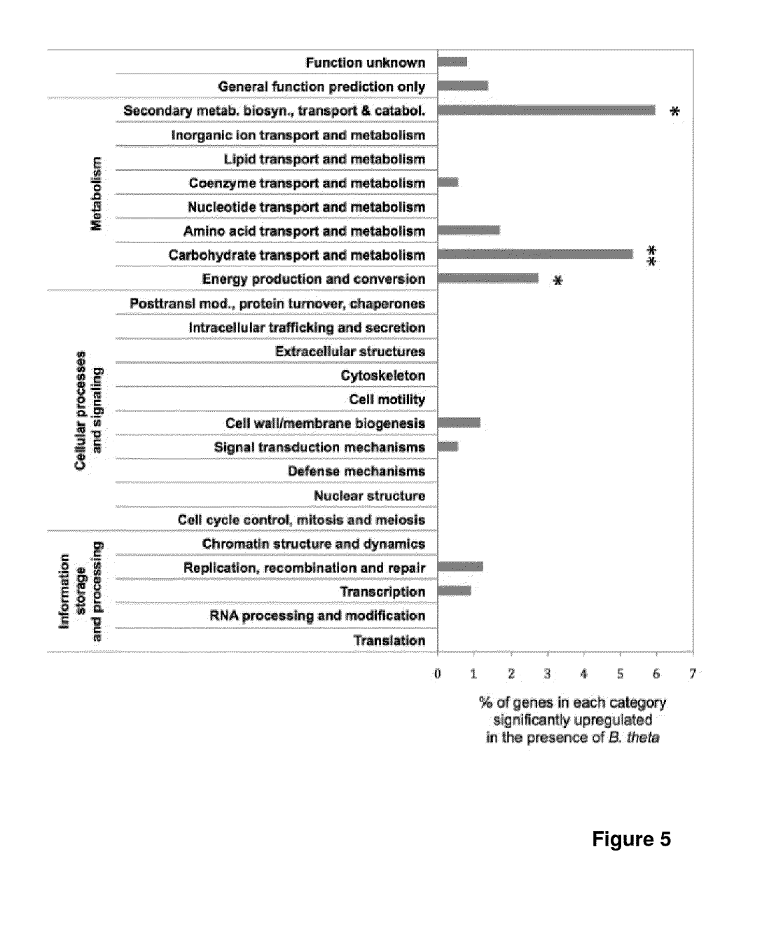

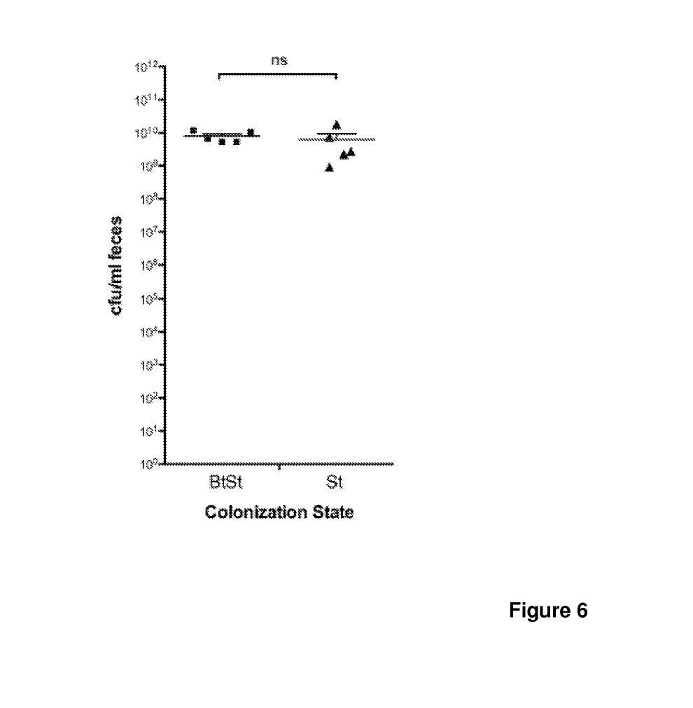

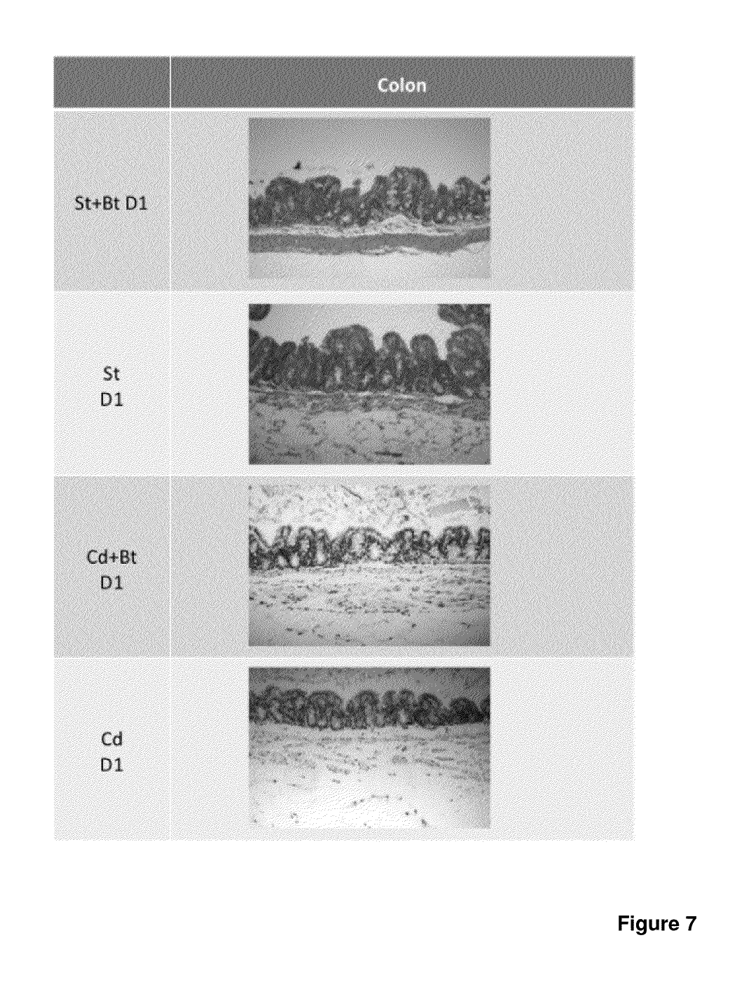

We used transcriptional profiling of Salmonella typhimurium from orally infected gnotobiotic mice to gain insight into the pathogen's biology while inhabiting the gastrointestinal tract. Mice that were monoassociated with the model gut symbiont Bacteroides thetaiotaomicron (Bt) were used as a simplified model of a microbiota that is susceptible to pathogen emergence. Five days after S. typhimurium infection of the Bt-monoassociated or germ-free (GF) mice (FIG. 1a), cecal contents were collected and subjected to transcriptional profiling using a custom S. typhimurium GeneChip. In the presence of Bt, all 59 S. typhimurium genes that displayed significantly altered expression relative to infection of GF mice were upregulated (Table 1). Functional classification of these genes revealed enriched COG categories: “carbohydrate metabolism and transport” and “secondary metabolites biosynthesis, transport, and catabolism” (FIG. 5). Genes encoding host mucin carbohydrate metabolism pathways are prominently represented in this gene set, including three operons encoding catabolic pathways for sialic acid, fucose, and the fucose catabolite propanediol, (nan, fuc and pdu, respectively) (FIG. 1b). We surveyed expression of genes within the nan and fuc operons 1 day after S. typhimurium infection in GF or Bt-monoassociated mice, to determine if these operons identified by expression profiling on day 5 post-infection also display high expression earlier in the infection. S. typhimurium nanE and fucI are significantly upregulated 1 day after infection of Bt-monoassociated mice relative to infection of GF mice (nanE, 6.0-fold, p=1.47×10⁻⁵; fucI, 3.5-fold, p=0.0028) (FIG. 1c) when S. typhimurium densities and host pathology are similar between colonization states (FIGS. 6 and 7). These data are consistent with S. typhimurium catabolizing sialic acid and fucose in the lumen of the gut in a Bt-dependent manner soon after infection.

TABLE 1

| | | | q- |
|---|---|---|---|
| | | Fold | value |
| Gene ID | Gene Name | Change | (%) |
| STM3335 | putative cytoplasmic protein yhcH | 5.4 | 0 |
| STM3337 | putative ManNAc6P epimerase COG3010G | 5.6 | 0 |
| | nanE | | |
| STM3338 | putative sialic acid transporter nanT | 4.3 | 0 |
| STM3339 | Nacetylneuraminate lyase COG0329E | 7.4 | 0 |
| | MnanA | | |
| STM3336 | Nacetylmannosamine kinase COG1940KG | 5.6 | 0 |
| | nanK | | |
| STM4045 | rhamnulose1phosphate aldolase COG0235G | 4.6 | 0 |
| | rhaD | | |
| STM2978 | putative Lfucosebinding protein COG4154G | 4.2 | 0 |
| | fucU | | |
| STM1614 | putative PTS system enzyme IIC component | 2.4 | 0 |
| | COG3775G STM1614 | | |
| STM0720 | putative glycosyl transferase COG1216R | 2 | 0 |
| | STM0720 | | |
| STM4046 | Lrhamnose isomerase rhaA | 8.1 | 0 |
| STM2977 | Lfuculokinase COG1070G fucK | 5.2 | 0 |
| STM0718 | putative cytoplasmic protein STM0718 | 2.6 | 0 |
| STM0710 | putative POT family transport protein | 2.1 | 0 |
| | COG3104E ybgH | | |
| STM0101 | Lribulose5phosphate 4epimerase COG0235G | 13.6 | 0 |
| | araD | | |
| STM1615 | putative nucleoside triphosphatase | 2.2 | 0 |
| | COG0434R STM1615 | | |
| STM3015 | putative aspartate racemase COG1794 | 3.2 | 0 |
| | MygeA | | |
| STM2044 | propanediol dehydratase reactivation protein | 4.1 | 0 |
| | pduH | | |
| STM2976 | Lfucose isomerase COG2407G fucI | 4.1 | 0 |
| STM2055 | polyhedral body protein pduU | 3.1 | 4.9 |
| STM0102 | Larabinose 11.2 COG2160G araA | 11.2 | 4.9 |
| STM2973 | L1,2propanediol oxidoreductase COG1454C | 4.7 | 4.9 |
| | fucO | | |
| STM0888 | arginine transport system component | 1.6 | 4.9 |
| | COG4160E artM | | |
| STM0717 | putative inner membrane protein STM0717 | 1.9 | 4.9 |
| STM0148 | putative cytoplasmic protein COG3940R | 3.5 | 4.9 |
| | STM0148 | | |
| STM3239 | putative transport protein COG0814E yhaO | 3 | 4.9 |
| STM4047 | rhamnulokinase COG1070G rhaB | 5.4 | 4.9 |
| STM1613 | putative PTS system enzymeIIB component | 2.9 | 4.9 |
| | COG3414G STM1613 | | |
| STM2974 | Lfuculose phosphate aldolase COG0235G | 4.2 | 4.9 |
| | fucA | | |
| STM1612 | putative cellulase protein COG1363G | 1.9 | 4.9 |
| | STM1612 | | |
| STM1616 | putative sugarspecific PTS enzyme II | 2 | 4.9 |
| | COG1762GT STM1616 | | |
| STM2043 | propanediol dehydratase reactivation protein | 4.5 | 4.9 |
| | pduG | | |
| STM0685 | Nacetylglucosaminespecific enzyme IIABC | 2.4 | 4.9 |
| | COG1263G nagE | | |
| STM2052 | propanol dehydrogenase COG1454C pduQ | 3.4 | 4.9 |
| STM2057 | acetate/propionate kinase COG0282C pduW | 3.4 | 4.9 |
| STM2938 | putative cytoplasmic protein COG1518L | 1.5 | 4.9 |
| | STM2938 | | |
| STM0719 | putative UDPgalactopyranose mutase | 1.6 | 4.9 |
| | COG0562M STM0719 | | |
| STM2042 | propanediol dehydratase small subunit | 3.8 | 4.9 |
| | COG4910Q pduE | | |
| STM0725 | putative glycosyltransferase STM0725 | 2 | 5.7 |
| STM2047 | propanediol utilization protein pduL | 3.3 | 5.7 |
| STM2048 | propanediol utilization protein pduM | 3 | 5.7 |
| STM2049 | polyhedral body protein pduN | 2.9 | 5.7 |
| STM2051 | CoAdependent propionaldehyde | 3.5 | 5.7 |
| | dehydrogenase COG1012C pduP | | |
| STM0724 | putative glycosyltransferase COG1216R | 1.5 | 5.7 |
| | STM0724 STM1618 | | |
| STM1618 | putative transcriptional represser of sgc | 1.8 | 6.8 |
| | operon COG1349KG | | |
| STM2041 | propanediol dehydratase medium subunit | 4.8 | 6.8 |
| | pduD | | |
| STM3679 | putative cytoplasmic protein COG3533S | 2.5 | 6.8 |
| | STM3679 | | |
| STM4048 | Lrhamnose operon regulatory protein rhaS | 2.5 | 6.8 |
| STM2053 | polyhedral body protein COG4656C pduS | 2.7 | 6.8 |
| STM2050 | propanediol utilization protein COG2096S, | 3 | 6.8 |
| | COG3193RpduO | | |
| STM2290 | putative transport protein yfaV | 1.4 | 6.8 |
| STM2054 | polyhedral body protein COG4577QC pduT | 2.5 | 7.4 |
| STM2056 | propanediol utilization protein COG4917E | 2.2 | 8.8 |
| | pduV | | |
| STM0394 | arabinose polymer transporter COG2814G | 2.9 | 8.8 |
| | araJ | | |
| STM3238 | putative inner membrane protein yhaN | 2.2 | 8.8 |
| STM0097 | DMA polymerase II COG0417L polB | 1.4 | 11.9 |
| STM0457 | putative hydrolase COG0561R cof | 2.6 | 11.9 |
| STM0103 | ribulokinase COG1069C araB | 7.9 | 11.9 |
| STM4130 | outer membrane receptor protein precursor | 1.4 | 11.9 |
| | COG4206H btuB | | |
| STM3767 | putative cytoplasmic protein STM3767 | 1.3 | 11.9 |

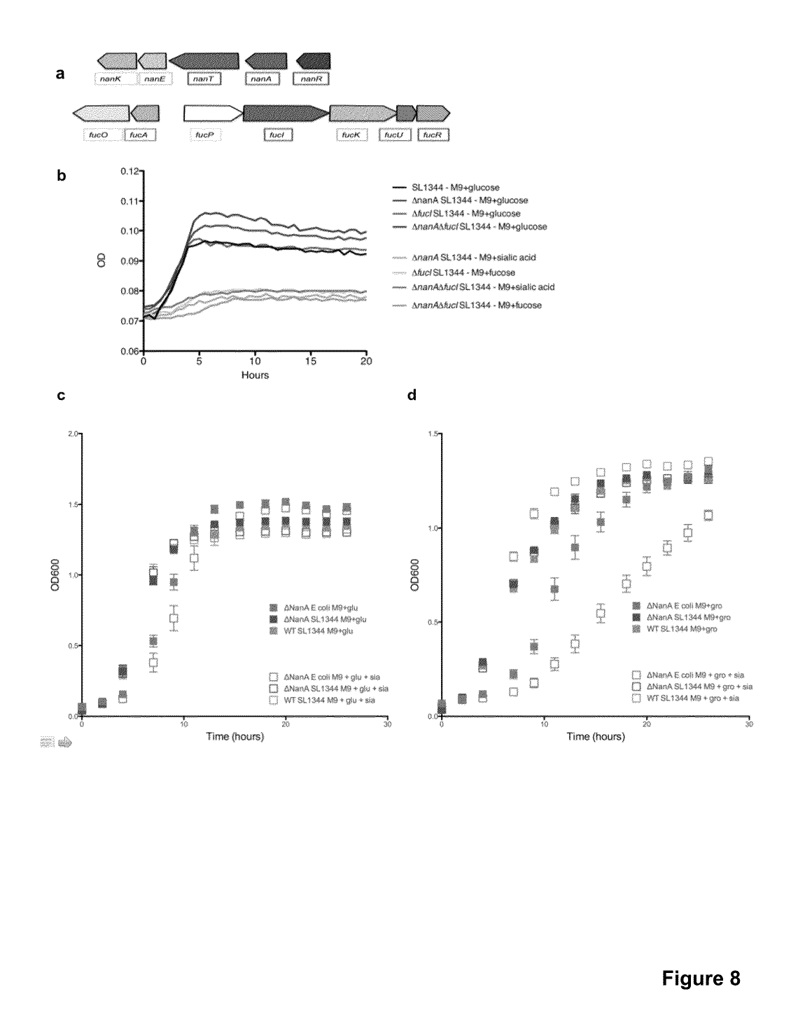

We next constructed mutant strains of S. typhimurium to quantitatively assess the requirement of sialic acid and fucose during emergence in vivo. Deletion of nanA and fucI, the first committed steps in the sialic acid and fucose utilization pathways, abolished growth of the strains on the respective sugars (FIG. 8). In competition experiments, Bt-monoassociated mice coinfected with wildtype S. typhimurium and a nanA/fucI double mutant strain (St-ΔnanAΔfucI) revealed a significant disadvantage of the mutant on days 1 and 2 after infection (day 1, CI=1.87, p=0.028; day 2, CI=1.45, p=0.016; FIG. 1d). This mutant, however, displayed no competitive disadvantage when competing with wild-type S. typhimurium within GF mice, consistent with S. typhimurium's sialic acid and fucose use being microbiota-dependent (day 1, p=0.26).

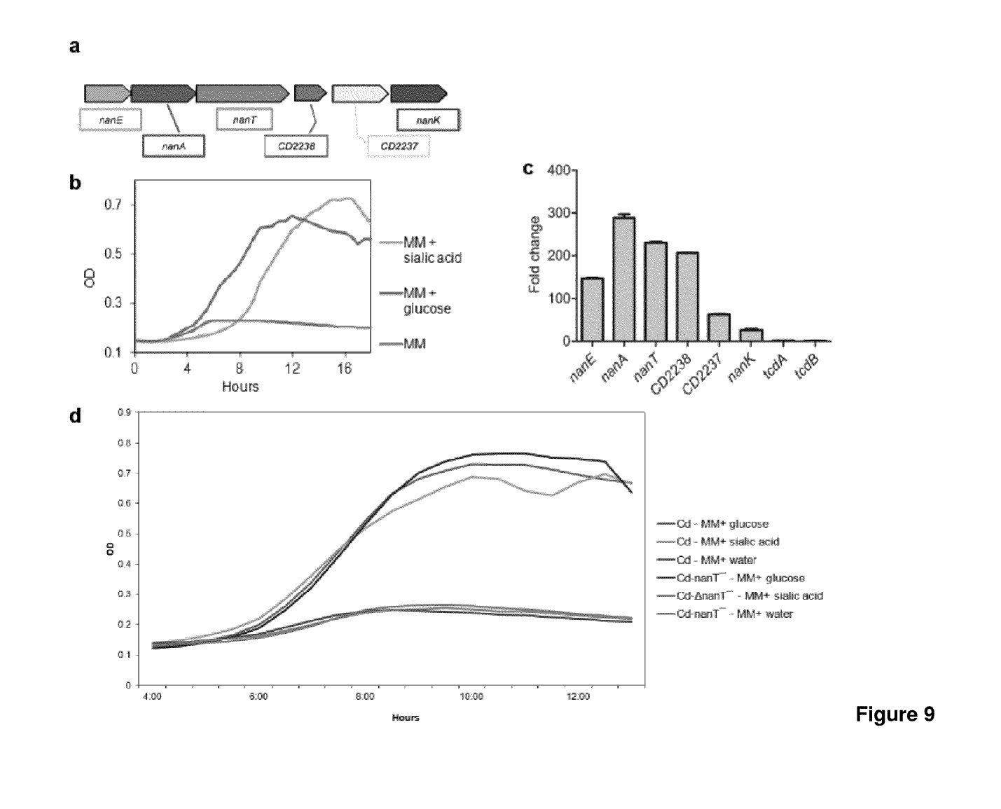

C. difficile possesses a sialic acid catabolic operon, like S. typhimurium, but encodes no apparent genes for fucose consumption (FIG. 9). To identify whether C. difficile also expresses sialic acid catabolism genes during its expansion within the gut, we quantified the expression of two genes within the nan operon, nanE and nanA, by qRT-PCR of RNA extracted from gnotobiotic mouse cecal contents. C. difficile nanE and nanA displayed elevated expression in Bt-monoassociated mice relative to expression levels observed when C. difficile colonized GF mice alone (nanE, 15-fold higher expression, p=0.02; nanA 11-fold higher expression, p=0.039; FIG. 1e). The presence of Bt in the gut of gnotobiotic mice resulted in an increased density of C. difficile one day post-infection compared to infection of GF mice (1.5×10⁸ vs. 7.9×10⁸ CFU/ml; p=0.0009; FIG. 1f).

Many commensal and pathogenic bacteria can utilize sialic acids from their hosts as a source of energy, carbon, and nitrogen. However, some bacteria, such as Bt, encode the sialidase required to cleave and release this terminal sugar from the mucosal glycoconjugates, but lack the catabolic pathway (i.e., nan operon) required to consume the liberated monosaccharide. Presumably, the release of sialic acids allows Bt to access highly coveted underlying carbohydrates in the mucus (Martens, E. C., et al. Cell host & microbe 4, 447-457 (2008)) (Marcobal, A. et al. J. Agric. Food Chem. 58, 5334-5340, (2010)). Conversely, S. typhimurium and C. difficile encode the nan operon but each lacks the sialidase required for sialic acid liberation (Hoyer, L. L., et al. Molecular microbiology 6, 873-884 (1992)) (Sebaihia, M. et al. Nature genetics 38, 779-786 (2006)).

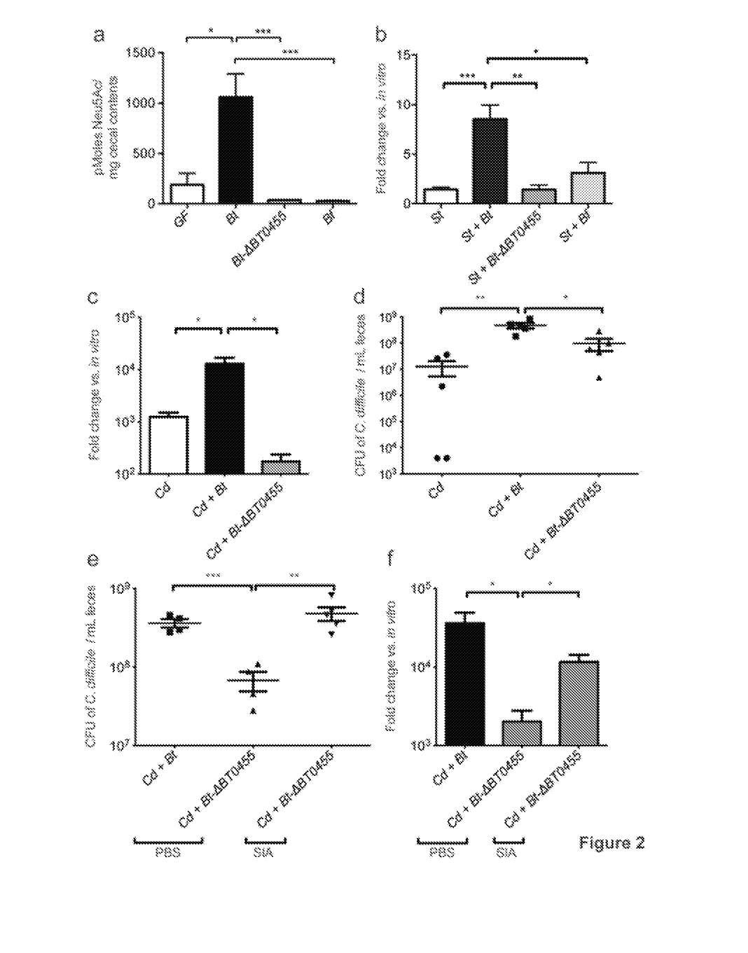

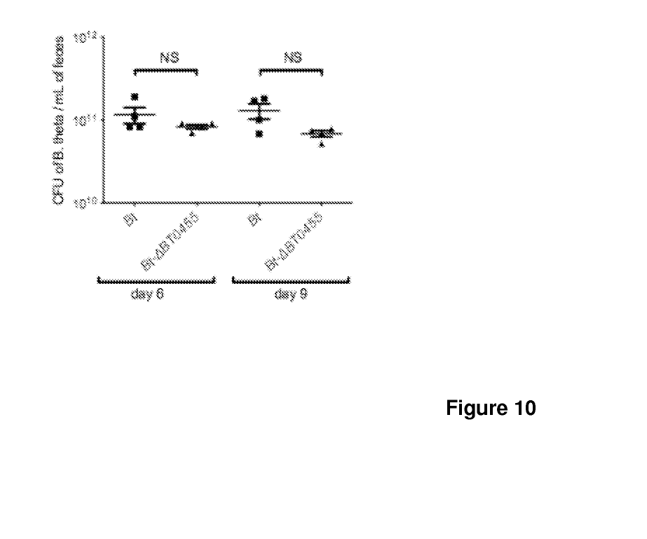

We quantified levels of free sialic acids in the ceca of Bt-monoassociated and GF mice. Bt-monoassociated mice exhibited a significantly higher concentration of the common sialic acid N-acetylneuraminic acid (Neu5Ac) versus GF mice, consistent with Bt's ability to liberate but not consume the monosaccharide (1059 pmoles/mg, Bt-associated; 188 pmoles/mg, GF; p=0.029; FIG. 2a). Colonization of mice with Bt-ΔBT0455 (a mutant strain of Bt lacking a predicted cell surface sialidase that achieves the same density as wt in vivo; FIG. 10) did not result in increased free sialic acid, nor did colonization with Bacteroides fragilis (Bt), which encodes both a sialidase and the nan operon and is therefore able to catabolize Neu5Ac (FIG. 2a). Expression of S. typhimurium's nan operon was reduced upon infection of gnotobiotic mice colonized with Bt-ΔBT0455 or B. fragilis, consistent with S. typhimurium's dependence upon elevated levels of microbiota liberated sialic acid (FIG. 2b).

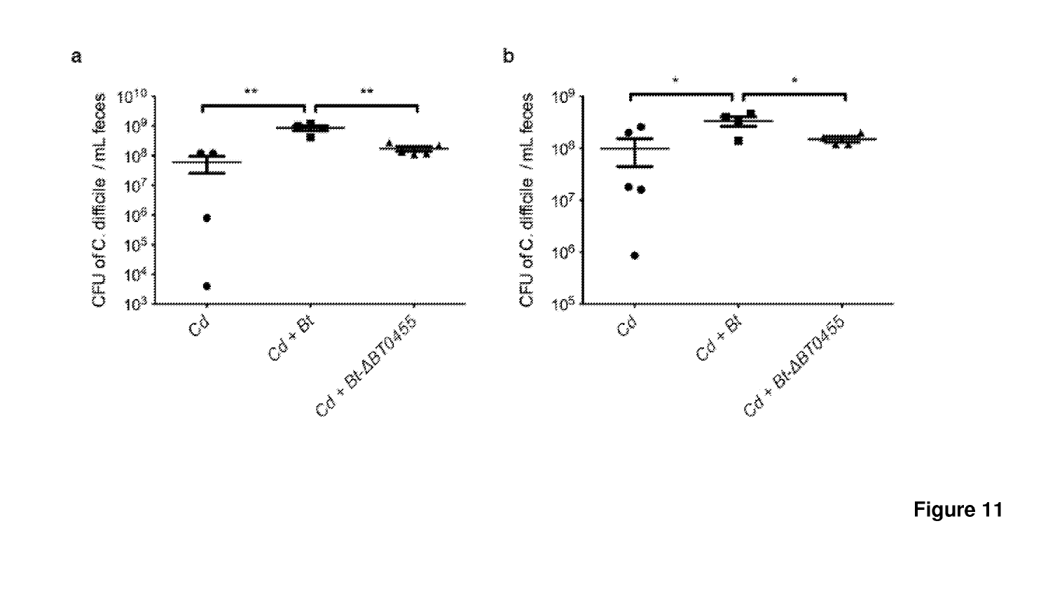

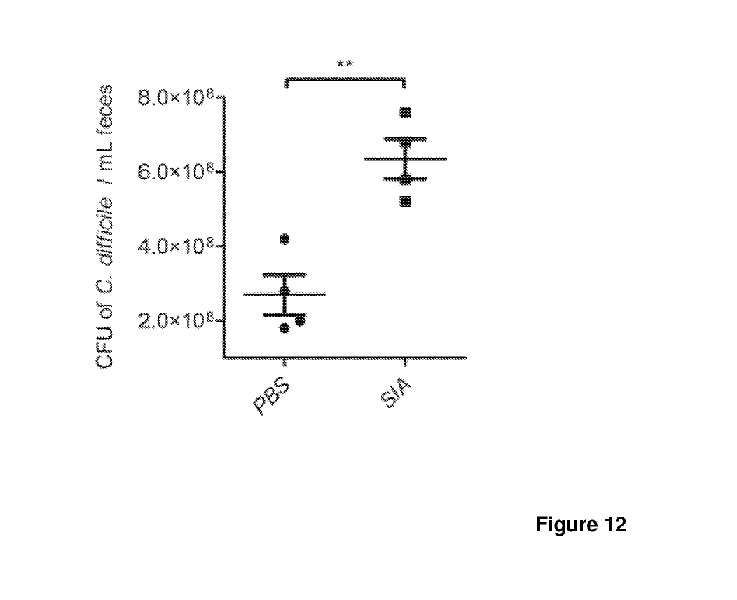

Loss of Bt-liberated sialic acid impacts C. difficile in a manner similar to that observed with S. typhimurium. Nan gene expression in C. difficile was lower in mice colonized with the sialidase-deficient mutant Bt-ΔBT0455 relative to expression in the presence of Bt-colonized mice (nanE, 75-fold higher expression, p=0.0187; FIG. 2c). Furthermore, C. difficile density decreased in infected mice colonized with Bt-ΔBT0455 mutant relative to densities in mice colonized with wild-type Bt, (9.7×10⁷ vs. 4.6×10⁸ CFU/ml; p=0.0143) illustrating the importance of Bt-liberated sialic acid in C. difficile emergence in vivo (FIG. 2d; FIG. 11). Free sialic acid was orally administered to Bt-ΔBT0455 and C. difficile co-colonized mice to determine if exogenous administration of the monosaccharide could reverse the decrease in C. difficile density by complementing the sialidase deficiency in this model. C. difficile densities increased 1 day post-infection in Bt-ΔBT0455 monoassociated mice fed free sialic acid compared to unsupplemented controls (4.8×10⁸ vs. 6.8×10⁷ CFU/ml; p=0.0066) reaching densities similar to those observed in the presence of wild-type Bt (FIG. 2e). Furthermore, expression of C. difficile nanE increases in the sialic acid-fed Bt-ΔBT0455-associated mice, further demonstrating that sialic acid use by C. difficile occurs concomitant with its increased densities in vivo (nanE, 58-fold higher expression over PBS-treated controls, p=0.019; FIG. 2f). Notably, free sialic acid administration to GF mice infected with C. difficile resulted in higher densities of the pathogen in the lumen of the gut, confirming the important role of this monosaccharide in vivo (FIG. 12). These data demonstrate that sialic acid catabolism by C. difficile promotes higher densities of the pathogen and depends upon the availability of the liberated monosaccharide within the lumen of the gut.

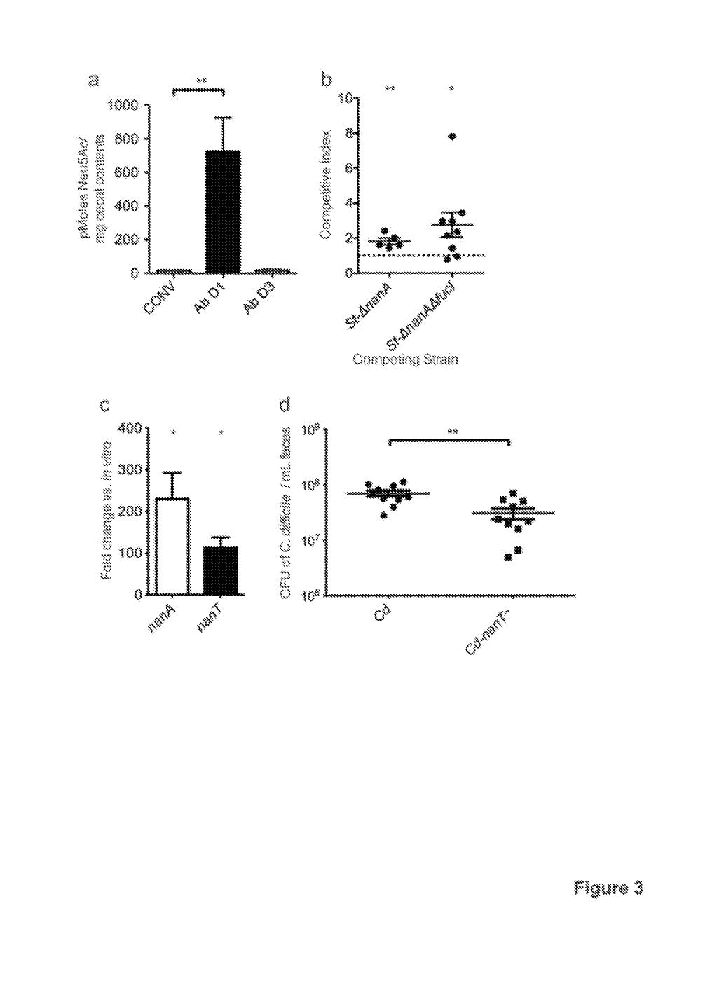

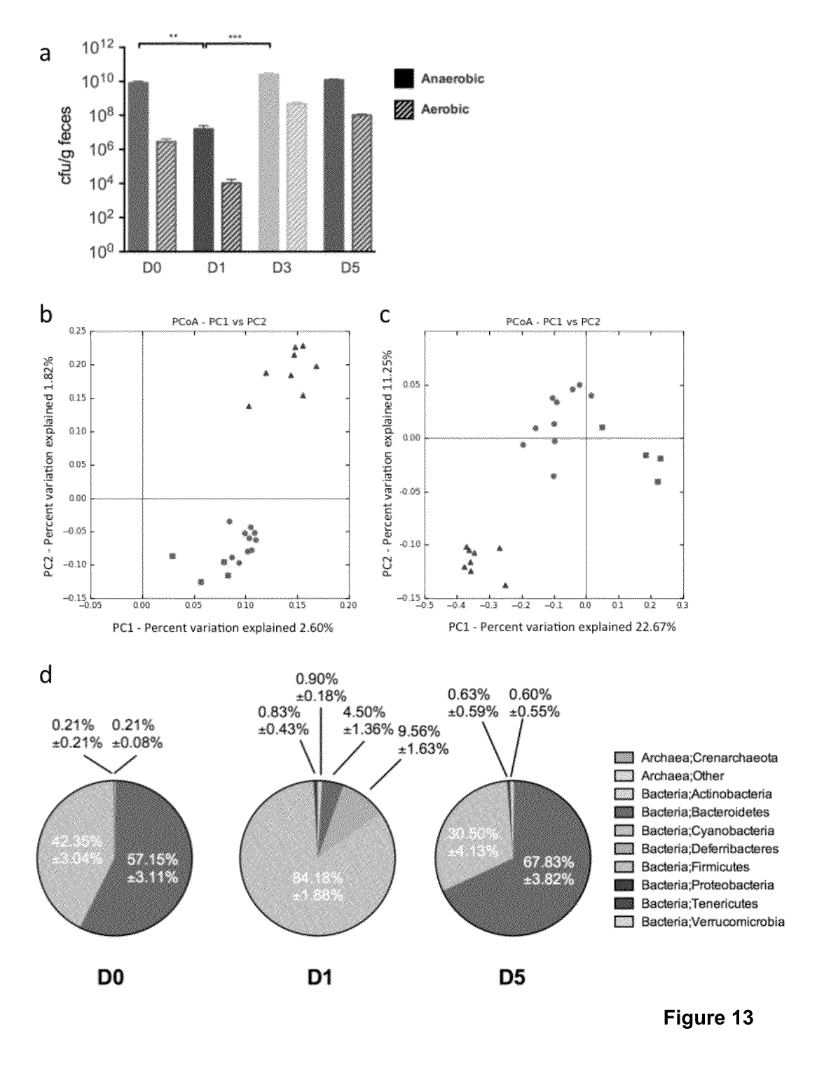

To determine if sialic acid use is relevant to pathogen emergence in an antibiotic-treated complex microbiota, we quantified free sialic acids in the ceca of conventional mice before and after antibiotic treatment. Levels of free Neu5Ac were very low within untreated conventional mice, consistent with efficient partitioning of Neu5Ac between members of an undisturbed complex microbiota (FIG. 3a). However, antibiotic-treated mice exhibited elevated levels of free sialic acid 1 day after treatment (725 pmoles/mg 1 day post-streptomycin compared to 17 pmoles/mg in untreated mice; p=0.0019), a time frame that coincides with pathogen emergence and acute microbiota disturbance (FIG. 13) (Stecher, B. et al. PLoS Biol 5, 2177-2189 (2007)). The pool of free sialic acids decreased by day 3 posttreatment, consistent with recovery of the microbiota after antibiotic treatment (Stecher, B. et al. PLoS Biol 5, 2177-2189 (2007)) (FIG. 3a). St-ΔnanA and St-ΔnanAΔfucI mutants both showed a competitive defect relative to wild-type St 1 day after infection in antibiotic-treated conventional mice (St-ΔnanA, CI=1.83 p=0.0095; St-ΔnanAΔfucI, CI=2.77, p=0.036), consistent with sialic acid and fucose utilization providing an advantage to S. typhimurium during emergence (FIG. 3b). To test whether C. difficile relies upon sialic acid catabolism in post-antibiotic emergence, we quantified the expression of the nan operon in antibiotic-treated conventional mice 1 day post-infection. Coincident with expansion of C. difficile, the nan operon was highly induced compared to basal expression in vitro (nanA, 230-fold, p=0.0358; nanT, 112-fold, p=0.0217) confirming that C. difficile expresses this operon at high levels during its post-antibiotic-emergence within a complex microbiota (FIG. 3c). As a test of sialic acid catabolism importance in C. difficile emergence, we constructed a nanT-mutant strain of C. difficile (Cd-nanT) that is deficient in sialic acid consumption (FIG. 7). Cd-nanT was significantly compromised in post-antibiotic emergence of conventional mice relative to wt Cd (3.1×10⁷ vs. 7.0×10⁷ CFU/ml; p=0.0023) demonstrating the importance of sialic acid catabolism to C. difficile in attaining high densities in the context of an antibiotic-disrupted complex microbiota (FIG. 3d).

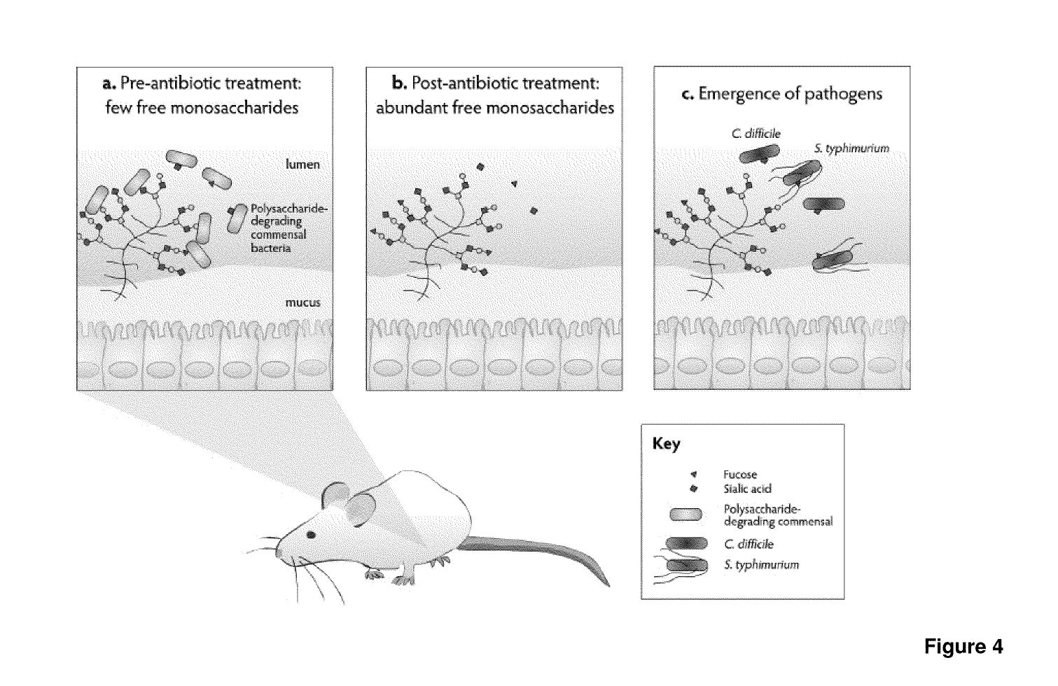

Recent studies have illustrated that enteric bacterial pathogens can subvert aspects of host inflammation to hold potential competitors within the microbiota at bay and enable pathogen proliferation (Stecher, B. et al. PLoS pathogens 6, e1000711 (2010)) (Winter, S. E. et al. Nature 467, 426-429 (2010)) (Lupp, C. et al. Cell host & microbe 2, 204 (2007)) (Barman, M. et al. Infection and immunity 76, 907-915 (2008)). Our results indicate that the antibiotic-associated pathogens S. typhimurium and C. difficile exploit increases in mucosal carbohydrate availability that occur upon disruption of the competitive ecosystem in which nutrients are typically efficiently consumed by endogenous community members. The transient post-antibiotic increase in monosaccharides liberated by the resident microbiota from host mucus provides a window of opportunity for these pathogens to expand to densities sufficient to induce self-promoting host inflammation (FIG. 4). Implicit in these findings are new potential therapeutic strategies to combat post-antibiotic pathogen emergence.

### Materials and Methods

Bacterial Strains and Culture Conditions.

B. thetaiotaomicron (VPI-5482) was grown in TYG medium. S. typhimurium (SL1344) was grown in LB. C. difficile (630) was grown in Reinforced Clostridial Medium (RCM) (Becton Dickinson, Md.). For strains and primers see Table 2.

TABLE 2

| Name | Primer used for generation | Notes |
|---|---|---|
| SL 1344 | n/a | Wild type |
| | | streptomycin-resistant |
| | | strain |
| ΔnanA::kan | ACAGGTATAAAGGTAGGTAGTTA | Strain is kanamycin |
| SL1344 | ATATATTCATCATCCGTAGAGGTA | resistant. |
| | GGTGTGTAGGCTGGAGCTGCTTC | |
| | GCTCACACTGTTGTAGGCCGGGC | |
| | AAGCGTAGCGCCCCCGGCATTAT | |
| | GCTGATTCCGGGGATCCGTCGAC | |
| ΔfucI::kan SL1344 | ACAAGCGGCGACAAACTGAACAT | Strain is kanamycin |
| | TTATCCGAATAACGTGAGGAATC | resistant. |
| | TGTCGTGTAGGCTGGAGCTGCTT | |
| | C | |
| | GTTCACGCGCCCGTAGTCCCGGT | |
| | AAACCTGCGCCACCGGCCTAAGC | |
| | CGCAACATATGAATATCCTCCTTA | |
| ΔnanA::kan | ACAAGCGGCGACAAACTGAACAT | Primers were used to |
| ΔfucI::cat SL1344 | TTATCCGAATAACGTGAGGAATC | generate |
| | TGTCGTGTAGGCTGGAGCTGCTT | fucI::cat LT2, which |
| | C | was then |
| | GTTCACGCGCCCGTAGTCCCGGT | transferred to |
| | AAACCTGCGCCACCGGCCTAAGC | nanA::kan SL1344 via |
| | CGCAACATATGAATATCCTCCTTA | P22 phage |
| | | transduction. |
| | | Strain is kanamyin |
| | | and |
| | | chloramphenicol- |
| | | resistant. |
| JIR8094 | n/a | Strain is erythromycin |
| | | sensitive. |
| Cd-nanT | IBS primer: | Strain is erythromycin |
| | AAAAAAGCTTATAATTATCCTTAG | resistant. |
| | TAACCCACCACGTGCGCCCAGAT | |
| | AGGGTG | |
| | EBS1d primer: | |
| | CAGATTGTACAAATGTGGTGATA | |
| | ACAGATAAGTCCACCACGGTAAC | |
| | TTACCTTTCTTTGT | |
| | EBS2 primer: | |
| | TGAACGCAAGTTTCTAATTTCGAT | |
| | TGTTACTCGATAGAGGAAAGTGT | |
| | CT | |
| | EBS universal primer: | |
| | CGAAATTAGAAACTTGCGTTCAG | |
| | TAAAC | |
| B. | n/a | |
| thetaiotaomicron | | |
| VPI 5482 | | |
| Bt-ΔBT0455 | BT0454.F.primary | |
| | GCGAACAGGAAGAATGTTCCG | |
| | BT0456.R.primary | |
| | GGAAACGGGAGTCAATAGCGGG | |
| | BT0454.F.secondary | |
| | GGCTGGATCCTCCCCAGGAATCA | |
| | CCG | |
| | BT0456.R.secondary | |
| | CCAATTTTATCTAGATAGAGGATG | |
| | CCG | |
| | BT0455.sew.F | |
| | AACTAATACCCCCATTTATGAAAA | |
| | GTCTTCGAATCTTTTTGG | |
| | BT0455.sew.R | |
| | CCAAAAAGATTCGAAGACTTTTCA | |
| | TAAATGGGGGTATTAGTT | |

Mice.

Germ-free Swiss-Webster mice were maintained in gnotobiotic isolators, in accordance with A-PLAC, the Stanford IACUC. Conventional Swiss-Webster mice (SWRF, Taconic) were used for antibiotic-treated experiments.

Expression Analyses.

Genome-wide transcriptional profiling of S. typhimurium in cecal contents was conducted using custom-made GeneChips. RMA-MS normalized signals were then analyzed for significant differences using SAM. For qRT-PCR, cDNA was synthesized from RNA purified from cecal contents or feces and analyzed using SYBR Green (ABgene) in a MX3000P thermocycler (Strategene). For primers see Table 3.

TABLE 3

| Gene | Forward Primer | Reverse Primer |
|---|---|---|
| ST nanE | TGCGTCGTTATACCATCCACAGA | ATCTGCCCTTAGTCAAAGCGTTG |
| ST nanK | ATCCTGCGTCATGCCGATAG | GAAAGCGACTACCGACTGCC |
| ST fucI | CGCCGCCTGTGAAGAAAAAT | GTAAACCGCCCCAGGACGCT |
| ST 16S | ATTGACGTTACCCGCAGAAGA | GGGATTTCACATCCGACTTGA |
| CD nanA | GTGTAGATGGGGCAATTGGT | ATCCAGCCTCAACACCTTGT |
| CD nanE | TTAGATGCTACCAATAGAGTTAGAC | TGAAACGCAATCTACACCGTA |
| | CA | |
| CD nanT | ATCAATGGGACTTGCAACAGT | CAACTGAATTAAGCCCTGTCG |
| CD nanK | GCAAGGTATGGTTGACCCAGT | CACCTCTACCTGCTCCAAGC |
| CD tcdA | TTCCCAACGGTCTAGTCCAA | TCATGGGATAGATATCAGGGCTA |
| CD tcdB | TGACCTCCAATCCAAACAAA | TGTTGCAATATTGGATGCTTT |
| CD 2237 | TGCTGCAATTCGTCTTGTTC | TGGATTCAAGGTGAAAGTGATG |
| CD 2238 | CGTTCACATCCATCAAGCAT | TTCAACATTGCATGAAGATTTACTT |
| CD 16S | AGAGTTTTACGACCCGAAGG | GCTAGTTGGTAAGGTAATGGCTTAC |

Quantification of Sialic Acids.

Free sialic acids were isolated from cecal content supernatants using a 1K MWCO filter and subjected to DMB derivitization prior to HPLC analysis.

Statistical Analyses.

The Student's t-test was used for statistical calculations, and * indicates p<0.05, ** indicates p<0.01 and *** indicates p<0.001. Error bars indicate SEM. n indicates the number of mice used per condition.

Bacterial Strains and Culture Conditions.

B. thetaiotaomicron (ATCC 29148, also known as VPI-5482), was grown anaerobically (6% H₂, 20% CO₂, 74% N₂) overnight in TYG medium (1% tryptone, 0.5% yeast extract, 0.2% glucose, w/v) supplemented with 100 mM potassium phosphate buffer, (pH 7.2), 4.1 mM cysteine, 200 μM histidine, 6.8 μM CaCl₂, 140 nM FeSO₄, 81 μM MgSO₄, 4.8 mM NaHCO₃, 1.4 mM NaCl, 1.9 μM hematin, plus 5.8 μM Vitamin K₃.

All strains of S. typhimurium were derived from wild-type strain SL1344, which is naturally streptomycin-resistant. Using the methods of Datsenko and Wanner (Datsenko, K. A., et al. Proc Natl Acad Sci USA, 2000. 97(12): p. 6640-5), mutant strains were first constructed in strain LT2, verified by PCR and then transduced into SL1344 using P22 phage transduction. Mutant strains and primers utilized in their generation are listed in Table 2. Growth defects were not observed on glucose for either mutant, and the presence of sialic acid did not pose a toxicity issue with the nanA mutant as has been previously reported for E. coli (Vimr, E. R., et al. Journal of Bacteriology, 1985. 164(2):845-853). For colonization experiments, S. typhimurium strains were grown in Luria-Bertani (LB) broth at 37° C. with aeration or on LB agar plates, with the appropriate antibiotics (200 μg/ml streptomycin, 30 μg/ml kanamycin). Minimal medium used for transcriptional profiling consisted of 100 mM KH₂PO₄ (pH 7.2), 15 mM NaCl, 8.5 mM (NH₄)₂SO₄, 4 mM L-cysteine, 1.9 mM hematin+200 mM L-histidine, 100 mM MgCl₂, 1.4 mM FeSO₄, 50 mM CaCl₂, 1 mg ml⁻¹ vitamin K₃, and 5 ng ml⁻¹ vitamin B₁₂, and 0.5% glucose (w/v). For evaluation of growth on various monosaccharides, strains were grown in M9 minimal media supplemented with 0.02% w/v histidine. Fecal densities (CFU) of S. typhimurium were quantified by duplicate sampling with 1 μl loops, and subsequent dilution and spot plating on plain LB agar for gnotobiotic experiments and LB agar with streptomycin for conventional experiments.

C. difficile strain 630 was utilized in all C. difficile experiments and was cultured in Reinforced Clostridial Medium (RCM)+cysteine (Becton Dickinson, Md.) anaerobically (6% H₂, 20% CO₂, 74% N₂). C. difficile growth curves were generated using minimal medium (MM) composed of ammonium sulfate, sodium carbonate, calcium chloride, magnesium chloride, manganese chloride, cobalt chloride, histidine hematin, vitamin B₁₂, vitamin K₁, FeSO₄, and 1% Bacto Tryptone diluted 1:1 with 1% or 0.5% carbon source. OD₆₀₀ was monitored using a BioTek PowerWave 340 plate reader

(BioTek, Winooski, Vt.) every 30 minutes, at 37° C. anaerobically (6% H₂, 20% CO₂, 74% N₂). Fecal densities (CFU) of C. difficile were quantified by duplicate sampling with 1 μl loops and subsequent dilution and spot plating on blood-BHI supplemented with erythromycin.

To construct the nanT null mutant (Cd-nanT), the ClosTron method for targeted gene disruption in C. difficile and detailed protocol were used (Heap, J. T., et al. J Microbiol Methods, 2010. 80(1): p. 49-55) (Adams, C. M., et al. PLoS Pathogens, In press). SOEing PCRs with primers IBS, EBS1d, EBS2 and EBS (see Table 2) were used to assemble and amplify the product for intron targeting, as outlined in the TargeTron users' manual (Sigma Aldrich). The retargeting sequence was digested with BsrGI/HindIII and cloned into pMTL007C-E2. The resulting plasmid was transformed into HB101/pRK24 for conjugation into JIR8094 (O'Connor, J. R., et al. Mol Microbiol, 2006. 61(5): p. 1335-51) (a generous gift from Aimee Shen) to generate CdnanT.

Reagents and Mice.

Germ-free Swiss-Webster mice were maintained in gnotobiotic isolators and fed an autoclaved standard diet (Purina LabDiet 5K67) or a polysaccharide-deficient diet (Sonnenburg, E. D., et al. Cell, 2010. 141(7): p. 1241-52), in accordance with A-PLAC, the Stanford IACUC. For all experiments involving C. difficile colonization of germ-free mice, the diet was switched to polysaccharide-deficient chow one day before inoculation with C. difficile.

Conventional Swiss-Webster mice (RFSW, Taconic) were used for S. typhimurium and C. difficile antibiotic-treated experiments.

Conventional mice were orally gavaged with 20 mg streptomycin dissolved in water 24 hours before infection, and starved 18 hours before infection. Mice were infected via oral gavage of 14 hour overnight cultures of S. typhimurium resuspended in PBS. For single infections of gnotobiotic mice, 10⁸ cfu of S. typhimurium were gavaged. For S. typhimurium competitive index experiments, pure cultures of wild-type and mutant bacteria were diluted to equal densities, mixed in a 1:1 ratio and serial diluted in PBS to a total of 10³ cfu/200 μl. Each mouse was orally gavaged with 200 μl of this dilution. Throughout the experiment, fecal samples were taken and dilutions were plated on LB agar plates containing streptomycin, which allows for growth of both the wild-type and mutant strains. Colonies from these plates were then patched onto LB agar+kanamycin plates to determine the proportion of kanamycin-resistant (mutant) cells. With each sample, the ratio of kanamycin-sensitive (wild-type) bacteria to kanamycin-resistant (mutant) bacteria was divided by the kans/kanr ratio determined from the original inoculum to produce the competitive index. All competitive indices were determined for fecal samples with the exception of St-ΔnanA, which was surveyed in cecal contents.

For C. difficile experiments involving conventional mice, antibiotics were administered in the water for 3 days, starting 6 days before inoculation including: kanamycin (0.4 mg/mL), gentamycin (0.035 mg/mL), colistin (850 U/mL), metronidazole (0.215 mg/mL) and vancomycin (0.045 mg/mL) (Chen, X. et al. Gastroenterology 135, 1984-1992 (2008)). Mice were then switched to regular water for 2 days, and administered 1 mg of clindamycin by oral gavage 1 day before inoculation with C. difficile. Inoculations were by oral gavage at a density of 10⁸ CFU from overnight cultures.

For sialic acid administration experiments, N-acetylneuraminic acid (Calbiochem or Santa Cruz Biotechnology) was administered in the water at a 1% concentration. Additionally, mice were orally gavaged 1 mg of sialic acid twice a day. The amount of sialic acid in the cecal contents were calculated to equal approximately 700 pmoles/mg of cecal contents, which mirrors the average concentration of free sialic acids we quantified post-antibiotic treatment (725 pmoles/mg).

Expression Analysis.

Genome-wide transcriptional profiling of S. typhimurium was conducted using custom-made GeneChips, which contain probes for all annotated coding sequences for S. typhimurium LT2. RNA was purified from cecal contents and in vitro culture and cDNA was prepared, fragmented and labeled as described (Sonnenburg, J. L., et al. Science, 2005. 307(5717): p. 1955-9).

GeneChip data were RMA-MS normalized as described (Stevens, J. R., et al. Proceedings of Conference on Applied Statistics in Agriculture 2008: p. 47-62) and log 2 transformed. Statistical significance for differential gene expression was determined using Significance Analysis of Microarrays (SAM) (Tusher, V. G., et al. Proc Natl Acad Sci USA, 2001. 98(9): p. 5116-21). The delta parameter was adjusted to achieve a FDR nearest to 10%, and this delta value was used to select significantly-regulated genes.

qRT-PCR analysis was performed on RNA extracted from cecal or fecal contents by phenolchloroform extraction and bead beating. Superscript II (Invitrogen) was utilized to convert RNA to cDNA, and SYBR Green (ABgene) in a MX3000P thermocycler (Strategene) was utilized. Fold changes were normalized to in vitro growths in Minimal Medium containing 0.5% glucose (MM-G) for C. difficile and LB for S. typhimurium.

Quantification of Sialic Acids.

All steps were carried out at 4° C. to minimize enzymatic hydrolysis. Approximately 200 mg of flash-frozen cecal contents were weighed out and resuspended in 400 μl dH₂O, Samples were vortexed for 30 minutes at max speed and centrifuged for 15 minutes at 14,000×g in the tabletop centrifuge.

The supernatant was stored, and the pellet resuspended in an additional 400 μl dH₂O. The tubes were vortexed individually until the pellet was dispersed, and then all samples were vortexed for 30 minutes, centrifuged, and supernatants were pooled. This process was repeated once more for a total volume of approximately 1 ml. 700 μl of each sample was filtered through a Pall 1K MWCO filter for 9 hours at 7,000×g. Samples were derivatized with DMB (1,2-diamino-4,5-methylene-dioxybenzene) as described previously (Manzi, A. E., et al. Anal Biochem, 1990. 188(1): p. 20-32). The resulting product was analyzed by reverse-phase HPLC using a C18 column (Dionex) at a flow rate of 0.9 ml/min, using a gradient of 5% to 11% acetonitrile in 7% methanol. The excitation and emission were 373 and 448 nm, respectively. The DMB-derivatized sialic acids were identified and quantified by comparing elution times and peak areas to known standards.

16S rRNA Microbial Community Composition Analysis.

Fecal DNA was isolated and amplicons generated of the 16S rRNA V4 region (515F, 806R). Samples were sequenced at Medical Genome Facility, Mayo Clinic, Rochester, Minn. using the MiSeq (Illumina) platform (Caporaso, J. G., et al. ISME J, 2012. 6(8): p. 1621-4). Data analysis was done using QIIME (Caporaso, J. G., et al. Nat Methods, 2010. 7(5): p. 335-6). Single end reads were analyzed to determine OTUs (Operational Taxonomic Units) at 97% sequence similarity using uclust. Taxonomy was assigned using RDP classifier against the GreenGenes database and a phylogenetic tree was built using FastTree. The OTU table was rarified to a sequencing depth of 900 for each set of samples. Beta diversity was determined using unweighted and weighted UniFrac (Lozupone, C., et al. BMC Bioinformatics, 2006. 7: p. 371).

Statistical Analyses.

The Student's t-test was used for statistical calculations, and * indicates p<0.05, ** indicates p<0.01 and *** indicates p<0.001. Error bars indicate SEM. n indicates the number of mice used per condition.

1. A method of reducing post-antibiotic growth of a pathogen, the method comprising:
administering an effective dose of an agent that limits altered nutrient availability in the gut following antibiotic-induced disruption of the normal microbiota.

2. The method of claim 1, wherein the pathogen is an enteric pathogen.

3. The method of claim 1, wherein the agent is orally administered.

4. The method of claim 1, wherein the altered nutrient is a cleavage product of a mucosal sugar.

5. The method of claim 4, wherein the altered nutrient is one or more of sialic acid and fucose.

6. The method of claim 1, wherein the pathogen is one or more of Clostridium difficile, Salmonella typhimurium, and Escherichia coli.

7. The method of claim 1, wherein the effective dose of an agent that limits altered nutrient availability is administered at initiation of antibiotic treatment.

8. The method of claim 1, wherein the effective dose of an agent that limits altered nutrient availability is administered after initiation of antibiotic treatment.

9. The method of claim 8, wherein the individual has been diagnosed with an enteric pathogen infection.

10. The method of claim 8, wherein the individual is at risk of infection with an enteric bacterial pathogen.

11. The method of claim 1, wherein the agent that limits altered nutrient availability is an enzyme inhibitor.

12. The method of claim 11, wherein the inhibitor is a fucosidase inhibitor.

13. The method of claim 11, wherein the inhibitor is a sialidase inhibitor.

14. The method of claim 13, wherein the sialidase inhibitor is selective or specific for sialidase activity present in the gut following antibiotic treatment.

15. The method of claim 14, wherein the sialidase comprises a commensal sialidase activity.

16. The method of claim 13, wherein the sialidase inhibitor is selected or formulated to have low or substantially absent systemic bioavailability.

17. The method of claim 16, wherein the sialidase inhibitor is orally administered.

18. The method of claim 13, wherein the sialidase inhibitor is selected from oseltamivir, zanamivir, laninamivir, and peramivir.

19. The method of claim 1, wherein the agent that limits altered nutrient availability is one or both of a prebiotic and a probiotic.

20. The method of claim 1, wherein the agent that limits altered nutrient availability inhibits the enteric pathogen use of the nutrient.

21. The method of claim 20, wherein the agent is one or more of an inhibitor of nutrient uptake and metabolism, an enzyme that degrades the nutrient.

22. The method of claim 21, wherein the agent is selected from sialic acid-use inhibitor, fucose-use inhibitor, and enzyme that degrades free sialic acid.

23. The method of claim 1, wherein two or more agents of differing activity are combined.

24. The method of claim 23, wherein the agents are the same class.

25. The method of claim 23, wherein the agents are of a different class.

26. The method of claim 1, wherein the effective dose of an agent that limits altered nutrient availability is combined in a formulation with an antibiotic.

27. A therapeutic formulation for use in the method of claim 1.

28. The method of claim 9, wherein the pathogen is C. difficile.

29. A method of treating a patient in need thereof with high enteric levels of sialic acid or fucose, the method comprising:
administering to said patient a therapeutic formulation as set forth in claim 27.


SEQUENCE LISTING (39 sequences)

SEQ ID NO: 1 - DNA, 139 bp, Artificial Sequence
  Synthetic nucleic acid sequence
acaggtataaaggtaggtagttaatatattcatcatccgtagaggtaggtgtgtaggctggagctgcttcgctcacactgttgtaggccgggcaagcgtagcgcccccggcattatgctgattccggggatccgtcgac

SEQ ID NO: 2 - DNA, 140 bp, Artificial Sequence
  Synthetic nucleic acid sequence
acaagcggcgacaaactgaacatttatccgaataacgtgaggaatctgtcgtgtaggctggagctgcttcgttcacgcgcccgtagtcccggtaaacctgcgccaccggcctaagccgcaacatatgaatatcctcctta

SEQ ID NO: 3 - DNA, 140 bp, Artificial Sequence; identical to SEQ ID NO: 2
  Synthetic nucleic acid sequence

SEQ ID NO: 4 - DNA, 53 bp, Artificial sequence
  Synthetic nucleic acid sequence
aaaaaagcttataattatccttagtaacccaccacgtgcgcccagatagggtg

SEQ ID NO: 5 - DNA, 60 bp, Artificial sequence
  Synthetic nucleic acid sequence
cagattgtacaaatgtggtgataacagataagtccaccacggtaacttacctttctttgt

SEQ ID NO: 6 - DNA, 49 bp, Artificial sequence
  Synthetic nucleic acid sequence
tgaacgcaagtttctaatttcgattgttactcgatagaggaaagtgtct

SEQ ID NO: 7 - DNA, 28 bp, Artificial sequence
  Synthetic nucleic acid sequence
cgaaattagaaacttgcgttcagtaaac

SEQ ID NO: 8 - DNA, 21 bp, Artificial sequence
  Synthetic nucleic acid sequence
gcgaacaggaagaatgttccg

SEQ ID NO: 9 - DNA, 22 bp, Artificial sequence
  Synthetic nucleic acid sequence
ggaaacgggagtcaatagcggg

SEQ ID NO: 10 - DNA, 26 bp, Artificial sequence
  Synthetic nucleic acid sequence
ggctggatcctccccaggaatcaccg

SEQ ID NO: 11 - DNA, 27 bp, Artificial sequence
  Synthetic nucleic acid sequence
ccaattttatctagatagaggatgccg

SEQ ID NO: 12 - DNA, 42 bp, Artificial sequence
  Synthetic nucleic acid sequence
aactaatacccccatttatgaaaagtcttcgaatctttttgg

SEQ ID NO: 13 - DNA, 42 bp, Artificial sequence
  Synthetic nucleic acid sequence
ccaaaaagattcgaagacttttcataaatgggggtattagtt

SEQ ID NO: 14 - DNA, 23 bp, Artificial sequence
  Synthetic nucleic acid sequence
tgcgtcgttataccatccacaga

SEQ ID NO: 15 - DNA, 23 bp, Artificial sequence
  Synthetic nucleic acid sequence
atctgcccttagtcaaagcgttg

SEQ ID NO: 16 - DNA, 20 bp, Artificial sequence
  Synthetic nucleic acid sequence
atcctgcgtcatgccgatag

SEQ ID NO: 17 - DNA, 20 bp, Artificial sequence
  Synthetic nucleic acid sequence
gaaagcgactaccgactgcc

SEQ ID NO: 18 - DNA, 20 bp, Artificial sequence
  Synthetic nucleic acid sequence
cgccgcctgtgaagaaaaat

SEQ ID NO: 19 - DNA, 20 bp, Artificial sequence
  Synthetic nucleic acid sequence
gtaaaccgccccaggacgct

SEQ ID NO: 20 - DNA, 21 bp, Artificial sequence
  Synthetic nucleic acid sequence
attgacgttacccgcagaaga

SEQ ID NO: 21 - DNA, 21 bp, Artificial sequence
  Synthetic nucleic acid sequence
gggatttcacatccgacttga

SEQ ID NO: 22 - DNA, 20 bp, Artificial sequence
  Synthetic nucleic acid sequence
gtgtagatggggcaattggt

SEQ ID NO: 23 - DNA, 20 bp, Artificial sequence
  Synthetic nucleic acid sequence
atccagcctcaacaccttgt

SEQ ID NO: 24 - DNA, 27 bp, Artificial sequence
  Synthetic nucleic acid sequence
ttagatgctaccaatagagttagacca

SEQ ID NO: 25 - DNA, 21 bp, Artificial sequence
  Synthetic nucleic acid sequence
tgaaacgcaatctacaccgta

SEQ ID NO: 26 - DNA, 21 bp, Artificial sequence
  Synthetic nucleic acid sequence
atcaatgggacttgcaacagt

SEQ ID NO: 27 - DNA, 21 bp, Artificial sequence
  Synthetic nucleic acid sequence
caactgaattaagccctgtcg

SEQ ID NO: 28 - DNA, 21 bp, Artificial sequence
  Synthetic nucleic acid sequence
gcaaggtatggttgacccagt

SEQ ID NO: 29 - DNA, 20 bp, Artificial sequence
  Synthetic nucleic acid sequence
cacctctacctgctccaagc

SEQ ID NO: 30 - DNA, 20 bp, Artificial sequence
  Synthetic nucleic acid sequence
ttcccaacggtctagtccaa

SEQ ID NO: 31 - DNA, 23 bp, Artificial sequence
  Synthetic nucleic acid sequence
tcatgggatagatatcagggcta

SEQ ID NO: 32 - DNA, 20 bp, Artificial sequence
  Synthetic nucleic acid sequence
tgacctccaatccaaacaaa

SEQ ID NO: 33 - DNA, 21 bp, Artificial sequence
  Synthetic nucleic acid sequence
tgttgcaatattggatgcttt

SEQ ID NO: 34 - DNA, 20 bp, Artificial sequence
  Synthetic nucleic acid sequence
tgctgcaattcgtcttgttc

SEQ ID NO: 35 - DNA, 22 bp, Artificial sequence
  Synthetic nucleic acid sequence
tggattcaaggtgaaagtgatg

SEQ ID NO: 36 - DNA, 20 bp, Artificial sequence
  Synthetic nucleic acid sequence
cgttcacatccatcaagcat

SEQ ID NO: 37 - DNA, 25 bp, Artificial sequence
  Synthetic nucleic acid sequence
ttcaacattgcatgaagatttactt

SEQ ID NO: 38 - DNA, 20 bp, Artificial sequence
  Synthetic nucleic acid sequence
agagttttacgacccgaagg

SEQ ID NO: 39 - DNA, 25 bp, Artificial sequence
  Synthetic nucleic acid sequence
gctagttggtaaggtaatggcttac